SEQUENCE LISTING (64 sequences)

SEQ ID NO: 1 - protein, 13 aa, Artificial Sequence
  Synthetic
LRWEPSSQPTIPI

SEQ ID NO: 2 - protein, 57 aa, Artificial Sequence
  Synthetic
VGIIAGLVLFGAVITGAVVAAVMWRRKSSDRKGGSYSQAASSDSAQGSDVSLTACKV

SEQ ID NO: 3 - protein, 17 aa, Artificial Sequence
  Synthetic
FTLTGLLGTLVTMGLLT

SEQ ID NO: 4 - protein, 9 aa, Artificial Sequence
  Synthetic
GVALQTMKQ

SEQ ID NO: 5 - protein, 9 aa, Artificial Sequence
  Synthetic
AARAVFLAL

SEQ ID NO: 6 - protein, 9 aa, Artificial Sequence
  Synthetic
SSKALQRPV

SEQ ID NO: 7 - protein, 9 aa, Artificial Sequence
  Synthetic
SYLDSGIHF

SEQ ID NO: 8 - protein, 10 aa, Artificial Sequence
  Synthetic
ACDPHSGHFV

SEQ ID NO: 9 - protein, 9 aa, Artificial Sequence
  Synthetic
YLSGANLNL

SEQ ID NO: 10 - protein, 9 aa, Artificial Sequence
  Synthetic
ETVSEQSNV

SEQ ID NO: 11 - protein, 9 aa, Artificial Sequence
  Synthetic
RIAECILGM

SEQ ID NO: 12 - protein, 9 aa, Artificial Sequence
  Synthetic
HLSTAFARV

SEQ ID NO: 13 - protein, 8 aa, Artificial Sequence
  Synthetic
YRPRPRRY

SEQ ID NO: 14 - protein, 9 aa, Artificial Sequence
  Synthetic
KTWGQYWQV

SEQ ID NO: 15 - protein, 9 aa, Artificial Sequence
  Synthetic
XLGTHTMEV

SEQ ID NO: 16 - protein, 9 aa, Artificial Sequence
  Synthetic
ITDQVPFSV

SEQ ID NO: 17 - protein, 9 aa, Artificial Sequence
  Synthetic
YLEPGPVTA

SEQ ID NO: 18 - protein, 10 aa, Artificial Sequence
  Synthetic
LLDGTATLRL

SEQ ID NO: 19 - protein, 10 aa, Artificial Sequence
  Synthetic
VLYRYGSFSV

SEQ ID NO: 20 - protein, 10 aa, Artificial Sequence
  Synthetic
SLADTNSLAV

SEQ ID NO: 21 - protein, 9 aa, Artificial Sequence
  Synthetic
RLMKQDFSV

SEQ ID NO: 22 - protein, 9 aa, Artificial Sequence
  Synthetic
RLPRIFCSC

SEQ ID NO: 23 - protein, 9 aa, Artificial Sequence
  Synthetic
LIYRRRLMK

SEQ ID NO: 24 - protein, 9 aa, Artificial Sequence
  Synthetic
ALLAVGATK

SEQ ID NO: 25 - protein, 10 aa, Artificial Sequence
  Synthetic
IALNFPGSQK

SEQ ID NO: 26 - protein, 9 aa, Artificial Sequence
  Synthetic
ALNFPGSQK

SEQ ID NO: 27 - protein, 9 aa, Artificial Sequence
  Synthetic
KIFGSLAFL

SEQ ID NO: 28 - protein, 9 aa, Artificial Sequence
  Synthetic
SPRWWPTCL

SEQ ID NO: 29 - protein, 9 aa, Artificial Sequence
  Synthetic
AEPINIQTW

SEQ ID NO: 30 - protein, 9 aa, Artificial Sequence
  Synthetic
EADPTGHSY

SEQ ID NO: 31 - protein, 9 aa, Artificial Sequence
  Synthetic
SLFRAVITK

SEQ ID NO: 32 - protein, 9 aa, Artificial Sequence
  Synthetic
EVDPIGHLY

SEQ ID NO: 33 - protein, 9 aa, Artificial Sequence
  Synthetic
FLWGPRALV

SEQ ID NO: 34 - protein, 9 aa, Artificial Sequence
  Synthetic
XAGIGILTV

SEQ ID NO: 35 - protein, 9 aa, Artificial Sequence
  Synthetic
STAPPVHNV

SEQ ID NO: 36 - protein, 10 aa, Artificial Sequence
  Synthetic
ILDTAGREEY

SEQ ID NO: 37 - protein, 9 aa, Artificial Sequence
  Synthetic
LLGRNSFEV

SEQ ID NO: 38 - protein, 10 aa, Artificial Sequence
  Synthetic
FLTPKKLQCV

SEQ ID NO: 39 - protein, 10 aa, Artificial Sequence
  Synthetic
VISNDVCAQV

SEQ ID NO: 40 - protein, 9 aa, Artificial Sequence
  Synthetic
ILAKFLHWL

SEQ ID NO: 41 - protein, 9 aa, Artificial Sequence
  Synthetic
MSLQRQFLR

SEQ ID NO: 42 - protein, 9 aa, Artificial Sequence
  Synthetic
LLGPGRPYR

SEQ ID NO: 43 - protein, 9 aa, Artificial Sequence
  Synthetic
SVYDFFVWL

SEQ ID NO: 44 - protein, 9 aa, Artificial Sequence
  Synthetic
TLDSQVMSL

SEQ ID NO: 45 - protein, 10 aa, Artificial Sequence
  Synthetic
EVISCKLIKR

SEQ ID NO: 46 - protein, 9 aa, Artificial Sequence
  Synthetic
KCDICTDEY

SEQ ID NO: 47 - protein, 10 aa, Artificial Sequence
  Synthetic
RGPGRAFVTI

SEQ ID NO: 48 - protein, 8 aa, Artificial Sequence
  Synthetic
SIINFEKL

SEQ ID NO: 49 - DNA, 225 bp, Artificial Sequence
  Synthetic
gagccctcgagctccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtga

SEQ ID NO: 50 - protein, 13 aa, Artificial Sequence
  Synthetic
LRWEPSSSTVSNM

SEQ ID NO: 51 - DNA, 39 bp, Artificial Sequence
  Synthetic
ctgagatgggagccctcgagctccactgtctccaacatg

SEQ ID NO: 52 - DNA, 30 bp, Artificial Sequence
  Synthetic
ccctcgagctccactgtctccaacatggcg

SEQ ID NO: 53 - DNA, 29 bp, Artificial Sequence
  Synthetic
cgcgcggccgcaagtccactccaggcagc

SEQ ID NO: 54 - DNA, 33 bp, Artificial Sequence
  Synthetic
gcgtctagagcttcagtcgtcagcatggctcgc

SEQ ID NO: 55 - DNA, 60 bp, Artificial Sequence
  Synthetic
ctgaccggcttgtatgctgtgtatgacttttttgtgtggctcggaggtggcggatccgcg

SEQ ID NO: 56 - DNA, 60 bp, Artificial Sequence
  Synthetic
cgcggatccgccacctccgagccacacaaaaaagtcatacacagcatacaagccggtcag

SEQ ID NO: 57 - DNA, 60 bp, Artificial Sequence
  Synthetic
ctgaccggcttgtatgctagtataatcaactttgaaaaactgggaggtggcggatccgcg

SEQ ID NO: 58 - DNA, 60 bp, Artificial Sequence
  Synthetic
cgcggatccgccacctcccagtttttcaaagttgattatactagcatacaagccggtcag

SEQ ID NO: 59 - DNA, 348 bp, Artificial Sequence
  Synthetic
ggatccggaggtggttctggtggaggttcgatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatgctgagatgggagccctcgagc

SEQ ID NO: 60 - DNA, 55 bp, Artificial Sequence
  Synthetic
gcgggatccggaggtggttctggtggaggttcgatccagaaaacccctcaaattc

SEQ ID NO: 61 - DNA, 45 bp, Artificial Sequence
  Synthetic
gcggctcgagggctcccatctcagcatgtctcgatcccagtagac

SEQ ID NO: 62 - DNA, 291 bp, Artificial Sequence
  Synthetic
gccctgctggtcattcctgtcgtgatgggcatcctcatcaccattttcggggtgtttctctatatcaaaaaggtggtcaagaaaccaaaggataatgagatgttaccccctgcggctcgacggcaagatccccaggagatggaagattatcccggtcataacaccgctgctccagtgcaggagacactgcacgggtgtcagcctgtcacacaggaggatggtaaagagagtcgcatctcagtgcaggagcggcaggtgacagacagcatagccttgaggcccctggtctga

SEQ ID NO: 63 - DNA, 46 bp, Artificial Sequence
  Synthetic
ccctcgagctccactgtctccaacatggccctgctggtcattcctg

SEQ ID NO: 64 - DNA, 29 bp, Artificial Sequence
  Synthetic
cgcgcggccgcggtcagcaagcagccatc

(19) United States
(12) Patent Application Publication
(10) Pub. No.: US 2006/0003315 A1
(43) Pub. Date: Jan. 5, 2006

(54) Membrane-anchored beta2 microglobulin covalently linked to mhc class 1 peptide epitopes

(75) Inventors: Gideon Gross, Korazim (IL); Alon Margalit, Western Galilee (IL)

Correspondence Address: BROWDY AND NEIMARK, P.L.L.C.;624 NINTH STREET, NW, SUITE 300, WASHINGTON, DC, 20001-5303, US

(73) Assignee: Gavish-Galilee Bio Applications Ltd., Kibbutz Glil Yam (IL)

(21) Appl. No.: 10/517,784

(22) PCT Filed: Jun. 12, 2003
(86) PCT No.: PCT/IL03/00501

§ 371 Date: Dec. 13, 2004

Related U.S. Application Data
(60) Provisional application No. 60/388,273, filed on Jun. 12, 2002.

(51) Int. Cl. C12Q 1/70; C12Q 1/68; C07H 21/04; C07K 14/16

(52) U.S. Cl. 435/5; 435/6; 435/69.3; 435/320.1; 435/325; 530/350; 536/23.72; 435/456

(57) ABSTRACT

The invention provides a polynucleotide comprising a sequence encoding a polypeptide comprising a beta2-microglobulin molecule linked through its carboxyl terminal to a polypeptide stretch that allows the anchorage of the beta2-microglobulin molecule to the cell membrane, and through its amino terminal to at least one antigenic peptide comprising a MHC class I epitope, wherein said antigenic peptide is not related to an autoimmune disease and is preferably derived from a tumor-associated antigen or from a pathogenic antigen. Antigen presenting cells, and DNA and cellular vaccines for treatment of cancer and infectious diseases, are also provided.

### FIELD OF THE INVENTION

The present invention is in the field of Immunology and relates to DNA molecules encoding chimeric polypeptides comprising β₂-microglobulin and a polypeptide stretch for anchoring the β₂-microglobulin molecule to the cell membrane, herein referred to as single-chimeric β₂-microglobulin (scβ₂m), and to such DNA molecules further comprising at least one antigenic peptide linked to the amino terminal of the β₂-microglobulin molecule, herein referred to as double-chimeric β₂-microglobulin (dcβ₂m), wherein the antigenic peptide is not a peptide related to an autoimmune disease, and to antigen-presenting cells expressing said scβ₂m and dcβ₂m polypeptides, as novel tools for efficient CTL induction for the treatment of cancer and infectious diseases.

ABBREVIATIONS: APC: antigen-presenting cell; β₂m: β₂-microglobulin; BCR: B cell receptor; CDR: complementarity-determining region; CTL: cytotoxic T lymphocyte; dcβ₂m: double-chimeric β₂-microglobulin; DC: dendritic cells; ER: endoplasmic reticulum; GPI: glycosyl-phosphatidylinositol; Ha: hemagglutinin; hβ₂m: human β₂-microglobulin; HLA: human leukocyte antigen (=human MHC); Ig: immunoglobulin; ITAM: immunoreceptor tyrosine-based activation motif; mAb: monoclonal antibody; mβ₂m: mouse β₂-microglobulin; MFI: mean fluorescence intensity; MHC: major histocompatibility complex; NP: nucleoprotein; OVA: chicken ovalbumin; RT-PCR: reverse transcriptase-polymerase chain reaction; scβ₂m: single-chimeric β₂-Microglobulin TAA: tumor associated antigen; TAP: transporter associated with antigen processing; TCR: T-cell receptor; T_{H}: T helper cells; TRP: tyrosinase-related protein.

### BACKGROUND OF THE INVENTION

The discovery, in recent years, of tumor associated antigens (TAAs) in a growing list of primary human tumors has led to the recognition that most, if not all types of human cancers express tumor antigens. The realization that some TAAs can elicit immune responses that lead to tumor rejection, has refueled interest in the field of cancer immunology, raising hopes for the development of potent anticancer immunotherapeutic tools and cancer vaccines (for reviews, see Minev et al., 1999; Gilboa et al., 1998; Rosenberg, 1999).

Tumor antigens can be divided according to the type of immune response they induce: humoral or cellular, which can be further subdivided into CD4⁺ (helper) and CD8⁺ (cytotoxic) T cell responses. Most TAAs known today were identified by their ability to induce cellular responses, predominantly by cytotoxic T lymphocytes (CTLs). CTLs utilize their clonotypic T cell receptor (TCR) to recognize antigenic peptides presented on major histocompatibility complex (MHC) class I molecules, which are expressed by most nucleated cells in the body. These proteins consist of a membrane-attached α heavy chain, which harbors three structurally distinct extracellular domains (α1-α3), and a non-covalently associated β₂ microglobulin (β₂m) light chain, that is not anchored to the cell membrane. Peptides, typically 8-10 amino acids long, bind to a special groove formed between the two membrane-distal domains of the α chain, α1 and α2, mainly via 2-3 dominant anchor residues.

CTLs serve as the major effector arm of the immune system and represent an important component of an animal's or an individual's immune response against a variety of pathogens and cancers. CTLs which have been specifically activated against a particular antigen are capable of killing the cell that contains or expresses the antigen. CTLs are particularly important in providing an effective immune response against intracellular pathogens, such as a wide variety of viruses, and some bacteria and parasites, as well as against tumors.

Some tumors down-regulate MHC class I expression, implying a strong selective pressure imposed by CTLs. In addition, CTLs are capable of recognizing single amino acid substitutions such as those that occur in TAAs resulting from point mutations. All these suggest that TAAs-derived MHC class I peptides are likely to constitute effective rejection antigens.

CTL activation, or priming, requires that antigenic peptides be presented initially on professional antigen-presenting cells (APCs), primarily dendritic cells (DCs), in secondary lymphoid organs (Steinman, 1989). In addition to highly efficient antigen presentation, DCs provide a co-stimulatory signal, which is mandatory for T cell priming, usually by engagement of their up-regulated B7 molecules with their CD28 receptor on the T cell (Janeway and Bottomly, 1994). Acquisition of the ability of the DC to prime CTLs is primarily mediated by antigen-specific CD4 T cells in a process referred to as ‘licensing’. It involves interaction of the TCR of the CD4 T cell with an antigenic peptide on an MHC class II molecule on the DC and concomitant engagement of the CD40 ligand (CD40L) on the T cell with the DC CD40 receptor (Guermonprez et al., 2002). Another unique feature of DCs is their ability to present peptides generated from exogenous proteins on their MHC class I molecules, a phenomenon generally referred to as cross-presentation (Heath and Carbone, 2001). Indeed, it is due to these unique properties, that autologous DCs are considered ideal for the induction of antitumor responses (for reviews, see Gilboa et al., 1998; Nouri-Shirazi et al., 2000; Chen et al., 2000; Porgador et al., 1996) and are thus widely explored as potential cancer vaccines.

Attempts to develop novel approaches for the generation of cancer vaccines have taken two major routes. Some approaches make use of the complete antigenic repertoire of the tumor cells. This is accomplished by induction of T cells by irradiated tumor cells, genetically modified to express cytokines, co-stimulatory molecules or foreign MHC, by pulsing of DCs with tumor-derived heat shock proteins, whole tumor cell extracts or total RNA (a minute amount of which can easily be amplified) and fusion of DCs with tumor cells (Zhang et al., 1997; Gong et al., 1997; Gong et al., 2000). These strategies are applicable to many types of tumors and, in theory, can induce a wide spectrum of antitumor CTLs. However, presentation of TAA-derived peptides of potential clinical benefit is not enriched and these protocols may thus fail to induce therapeutic CTLs (Sogn, 2000; Dalgleish, 2001). Furthermore, these procedures do not allow attribution of clinical response to particular antigens and, therefore, useful information cannot be deduced for broader implementation.

Other approaches for the generation of cancer vaccines are based on known TAAs. These include the design of peptide, DNA and recombinant viral vaccines, charging DCs with either purified tumor-associated proteins or TAA-derived peptides and presentation of TAA-derived peptides, which are produced following gene delivery into autologous or syngeneic (in mice) DCs (for review, see Gilboa et al., 1998).

Indeed, some encouraging data showing CTL induction and vaccine efficacy came from animal studies exploring either type of above-described approaches. However, clinical success in human trials has so far been limited, with little correlation between the observed number of specific anti-tumor CTLs and the actual clinical response (Sogn, 1998; Moingeon, 2001; Jager et al., 2002). This is attributed, in part, to requirement for help from CD4⁺ cells and to immunosuppressing cytokines produced by the tumor cells, but also to the fact that many of the identified MHC class I-associated TAA peptides are poorly presented on the cell surface because of low level of protein expression and low affinity for their restricting MHC class I molecule (Watson et al., 1995; Vora et al., 1997).

Intracellular proteins, as well as soluble protein antigens delivered into the cytoplasm of a cell, are degraded into short peptides by a cytosolic proteolytic system present in all cells. Those proteins targeted for proteolysis often have a small protein, called ubiquitin, attached covalently to a lysine-amino group near the amino terminal of the protein. These ubiquitin-protein complexes are degraded into a variety of peptides by a multifunctional protease complex called proteasome. Experimental evidence indicates that the immune system utilizes this general pathway of protein degradation to produce small peptides for presentation with class I MHC molecules. The peptides, generated in the cytosol by the proteasome, are translocated by a transporter protein, called TAP (for “transporter associated with antigen processing”), into the endoplasmic reticulum (ER), by a process that requires the hydrolysis of ATP. Within the ER membrane, newly synthesized class I α chain associates with calnexin until β₂m binds to the α chain. The class I α chain-β₂m heterodimer then binds to calreticulin and the TAP-associated protein tapasin. When a peptide delivered by TAP is bound to the class I molecule, folding of MHC class I is complete and it is released from the ER and transported to the surface of the cell. TAP has the highest affinity for peptides containing 8-13 amino acids. Peptides longer than the size required for MHC class I binding are further trimmed in the ER by assigned amino peptidases to acquire the optimal length.

A single cell can display thousands of different MHC class I bound peptides, most of them only at low frequency of less than 0.1% of the total. The density of MHC/peptide complexes on the cell surface determines the degree of T cell responsiveness (Levitsky et al., 1996; Tsomides et al., 1994; Gervois et al., 1996). CTL priming by a professional APC generally requires a higher density of specific complexes than that required on the surface of the target cell for activation of an armed effector CTL (Armstrong et al., 1998; Reis e Souza, 2001). The ability to generate high numbers of particular MHC class I/peptide complexes on the APC itself could, therefore, be of great value for elicitation of strong CTL responses, which may be effective against TAA-derived peptides of an otherwise limited distribution.

This realization has prompted attempts to enhance level of peptide presentation by APCs, either by increasing the intrinsic affinity of the peptide for the restricting MHC class I molecule, or by manipulations aiming at elevating the actual number of specific classI/peptide complexes on the cell surface. A recent study (Tirosh et al., 1999) has examined the effect of peptide affinity on CTL response it elicits, either by a chemical modification, which renders peptide binding to the class I groove irreversible, or by optimizing the MHC anchor residues of the peptide. Working with the TAP-deficient RMA-S cells, it was shown that improving the affinity of a murine TAA-derived peptide could indeed result in significant enhancement of CTL induction and inhibition of tumor growth. However, at least in this particular system, there seems to exist an affinity ceiling, beyond which a corresponding augmentation in the magnitude of the immune response could not be achieved. An important observation in this study is of a significant decrease in the initial number of specific complexes, both of low and high affinity peptides, which occurred in the first two hours post-loading. This finding underlines an inherent limitation associated with the transient nature of MHC-binding by exogenous antigenic peptides, and reinforces the prospects of genetic modification of DCs.

A number of studies have indeed attempted to increase the actual frequency of the desired antigenic class I complexes on the cell surface, through genetic engineering of improved class I-peptide ligands. For example, one group (Mottez et al., 1995; Lone et al., 1998) has constructed a chimeric MHC class I molecule, in which the antigenic peptide was covalently linked to the amino terminal of the α chain. These proteins were expressed on the surface of transfected cells and were capable of eliciting a specific CTL response. However, using this approach, each antigenic peptide should be constructed with its own restricting α chain. To overcome this problem, another group (Uger and Barber, 1998) has attached the antigenic peptide to the amino terminal of the monomorphic β₂m. Primary T cells from mice, which had been immunized with the specific peptide, could indeed selectively lyse transfected cells, expressing these constructs. However, the cells used for expression in this study were deficient in MHC class I expression, due to a TAP transporter mutation. Yet, in spite of lack of competition from cytosol-derived peptides, level of peptide presentation was limited. Using a similar design, another study (Tafuro et al., 2001) has recently demonstrated reconstitution of MHC class I presentation in human cancer cells, but these, again, were class I-negative, due either to a TAP defect or to lack of β₂m expression. Although a non-mutated lymphoblastoid cell line was also included in this study and potentiated specific CTL lysis, there is no evidence as to the actual level of peptide presentation in these cells.

Citation or identification of any reference in any section of this application shall not be construed as an admission that such reference is available as prior art to the present invention.

### SUMMARY OF THE INVENTION

The present invention relates, in one aspect, to a polynucleotide comprising a sequence encoding a polypeptide that is capable of high level presentation of antigenic peptides on antigen-presenting cells, wherein the polypeptide comprises a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope, wherein said antigenic peptide is not related to an autoimmune disease. This chimeric polypeptide is referred to herein as “double-chimeric β₂-microglobulin” (dcβ₂m).

In one embodiment, an epitope, which is an antigenic determinant of one sole antigen, is linked to the amino terminal of the β₂-microglobulin. In another embodiment, there are two or more epitopes that may be antigenic determinants of the same or of two or more different antigens. The epitopes/antigenic peptides may be derived from a tumor-associated antigen (TAA), from an infectious agent, e.g. a bacterial or viral protein, or they are TCR idiotypic peptides expressed by autoreactive T cells and BCR or antibody idiotypic peptides expressed by autoreactive B cells.

In another aspect, the present invention relates to a vector comprising a DNA molecule of the invention.

In a further aspect, the present invention relates to antigen-presenting cells (APCs), which express a dcβ₂m encoded by the DNA molecule of the invention as defined above. Any suitable professional APC can be used according to the invention such as dendritic cells, macrophages and B cells. In a preferred embodiment, the APC is a dendritic cell. Transfection or transduction of the cells is carried out by standard methods of recombinant DNA technology as well known to a person skilled in the art.

In one preferred embodiment, the APCs are capable of expressing a dcβ₂m polypeptide comprising at least one TAA peptide such as to present the TAA peptide(s) at a sufficiently high density to allow potent activation of peptide-specific cytotoxic T lymphocytes (CTL) capable of recognizing and binding to harmful tumor cells and causing their elimination or inactivation.

The present invention further provides a cancer vaccine comprising an agent selected from: (i) a DNA molecule encoding a dcβ₂m as defined herein wherein the at least one epitope linked to the amino terminal of β₂m is derived from at least one TAA; (ii) an expression vector comprising such DNA molecule (i); (iii) antigen presenting cells expressing a dcβ₂m as defined herein wherein the at least one epitope linked to the amino terminal of β₂m is derived from at least one TAA; (iv) antigen presenting cells (APCs) expressing a single-chimeric β₂-Microglobulin (scβ₂m) molecule as defined herein consisting of a β₂-microglobulin molecule linked through its carboxyl terminal to a polypeptide stretch that allows the anchorage of the β₂-microglobulin molecule to the cell membrane; and (v) APCs as defined in (iv) that have been pulsed with at least one TAA peptide.

The present invention still further provides pharmaceutical compositions for use in inducing a class I-restricted CTL response in a mammal comprising cells expressing a dcβ₂m of the invention.

### BRIEF DESCRIPTION OF THE FIGURES

FIGS. 1A-1B depict the construction and expression of dcβ₂m. FIG. 1A is a sketch of the dcβ₂m polypeptide associated on the cell surface with a MHC class I heavy (α) chain, while the antigenic peptide is in the binding groove. The transmembrane and cytoplasmic domains are derived from either the mouse CD3 ζ chain or the MHC class I heavy chain H-2K^{b}, and are covalently attached, via a short bridge, to the carboxyl teminus of human or mouse β₂m. The antigenic peptide is attached to the amino terminal of β₂m via a short linker with the sequence G₄S(G₃S)₂. FIG. 1B is a scheme of the genetic construct: pr, promoter; lead, leader peptide; p, antigenic peptide; li, linker peptide; br, bridge. Important restriction sites are indicated.

FIGS. 2A-2C show flow cytometry analysis of MD45 parental cells (FIG. 2A) and transfectants 425-44 (NP) cells (expressing NP₅₀₋₅₇ dcβ₂m) (FIG. 2B) and 427-44 (Ha) cells (expressing the Ha₂₅₅₋₂₆₂ dcβ₂m) (FIG. 2C). Cells were analyzed with primary antibodies against H-2K^{k} (clone AF3-12.1), hβ₂m (clone BM-63) and K^{k}/Ha₂₅₅₋₂₆₂ complex (Fab13.4.1) and detected with secondary goat anti-mouse IgG (Fab-specific)-FITC conjugated polyclonal antibodies.

FIG. 3 shows stimulation of the MD45 transfectants 425-44, 427-24 and 892S-36 (see Table 1 hereinafter) by different MHC-I allele-specific antibodies. Indicated cells at 5×10⁵/ml in 100 μl were incubated in wells of a microtiter plate pre-coated with the different antibodies at 5 μg/ml and then subjected to an in-cell X-Gal staining. Anti-K^{k} is AF3-12.1 and anti-K^{d} is SF1-1.1. Anti-TCR is the hamster anti-mouse CD3ε mAb 2C11, which served as a positive control for activation.

FIGS. 4A-4C show FACS analysis of RMA (FIG. 4A), RMA-S (FIG. 4B) and transfectant Y317-2 (expressing OVA₂₅₇₋₂₆₄ linked to human membranal β₂m) cells (FIG. 4C). Antibodies were: anti-H-2Db (28-14-8); anti-H-2K^{b} (20-8-4); anti-hβ₂m (BM-63) and anti-K^{b}-OVA₂₅₇₋₂₆₄ (25-D1.16). Cells were grown for 24 hours in serum-free medium prior to staining at both 27° C. and 37° C.

FIGS. 5A-5G show that a K^{b}/OVA₂₅₇₋₂₆₄-specific T cell hybridoma is activated by cells expressing OVA₂₅₇₋₂₆₄ dcβ₂m. B3Z cells, an H-2K^{b} restricted OVA₂₅₇₋₂₆₄-specific T cell hybridoma, were incubated with: 5A. no stimulation; 5B. Plastic-bound (5 μg/ml) anti-CD3ζ mAb (2C11); 5C. RMA cells; 5D. RMA cells loaded with synthetic OVA₂₅₇₋₂₆₄ at 2 μg/ml as a positive control; 5E. Y314-7 cells; 5F. Y317-2 cells; 5G. Y318-7 cells as a negative control. All cells were at 5×10⁵/ml. Cells were stained with X-Gal and visualized under a microscope.

FIGS. 6A-6B depict construction and expression of scβ₂m. FIG. 6A is a sketch of the scβ₂m polypeptide. The transmembrane and cytoplasmic domains are derived from either the mouse CD3 ζ chain or the MHC class I heavy chain H-2K^{b}, and are covalently attached, via a short bridge, to the carboxyl teminus of human or mouse β₂m. FIG. 6B is a scheme of the genetic construct: pr, promoter; lead, leader peptide; p, antigenic peptide; li, linker peptide; br, bridge. Important restriction sites are indicated.

FIG. 7 shows stabilization of MHC class I molecules by membranal β₂m. KD21-4 and KD21-6 RMA-S transfectants and parental RMA-S and RMA cells were grown in serum-free medium for 24 hours at 27° C. and 37° C. and then stained with anti-H-2D^{b} (28-14-8) and anti-hβ₂m (BM-63) mAbs. FACS analysis was performed with FACSCalibur (BD Biosciences).

FIG. 8 is a graph showing the ability of KD21-6 and D323-4 transfectants to bind exogenously added synthetic OVA₂₅₇₋₂₆₄ peptide through H-2K^{b}, in comparison with parental RMA-S cells. The cells were grown at 37° C. for 24 hours in serum-free medium and were then incubated for 42 hours with serial dilutions of synthetic OVA₂₅₇₋₂₆₄. Cells were stained with mAb 25.D1-16 and FACS analysis was performed with FACSCalibur. Mean fluorescence intensity was calculated using CellQuest software.

FIG. 9 is a graph showing generation of antigen specific CTLs following cell immunization. RMA-S and RMA-S/OVA (negative controls), RMA-S loaded with OVA₂₅₇₋₂₆₄ and RMA/OVA (positive controls), and transfectants Y317-2 and Y314-7 were injected i.p. twice at 10-day interval. Ten days after the second immunization, CTLs were prepared and the indicated cells (Y317-2, Y314-7 and RMA-S) were used as target cells in a cell cytotoxicity assay at effector/target ratio of 50:1. Histogram shows percent specific lysis.

FIG. 10 shows FACS analysis of RMA-S, KD21-6 and Y340-13 cells. Cells were analyzed with a primary antibody against hβ₂m (clone BM-63) and detected with secondary goat anti-mouse IgG (Fab-specific)-FITC conjugated polyclonal antibodies.

### DETAILED DESCRIPTION OF THE INVENTION

Duration of the functional MHC classI/peptide complex on the cell surface is governed by the affinity of the peptide for the MHC molecule. Dissociation of the peptide from its binding groove in the α heavy chain, results in practically irreversible disruption of the ternary complex formed between the α chain, β₂m and peptide. Both latter components are not anchored to the cell membrane and immediately detach from the cell, while the α chain is later internalized. Stabilization of a particular class I/peptide complex by enabling fast re-association is therefore likely to result in high level of presentation of the antigenic peptide.

In one aspect, the concept underlying the present invention is that connecting at least one epitope to one end (the amino terminal) of β₂m and anchoring this polypeptide to the cell membrane through its other end (the carboxyl terminal), will provide an exceedingly high level of the antigenic peptide directly to the ER in a TAP- and proteasome-independent manner and substantially increase complex stability, and consequently, the level of presentation of this peptide.

WO 01/91698 of the same applicants, hereby incorporated by reference in its entirety as if fully disclosed herein, discloses the development by genetic engineering of a novel MHC class I configuration, in which the β₂m light chain is anchored to the cell membrane, while harboring an antigenic peptide related to an autoimmune disease fused to its amino terminal. Expression of this construct results in an exceptionally high level of the MHC-peptide complex on the surface of transfected cells, despite competition from normally presented peptides. Thus, an influenza virus hemagglutinin-derived peptide (Ha₂₅₅₋₂₆₂), restricted by the mouse class I allele K^{k}, was linked to the amino terminal of β₂m by genetic engineering, while the carboxyl terminal was anchored to the membrane of transfected, K^{k}-expressing cells. Analyses performed with an anti-K^{k} mAb and another mAb, which shows exquisite specificity to the K^{k}/Ha₂₅₅₋₂₆₂ complex, revealed high levels of the complex on the surface of transfected cells. It should be emphasized that efficient pairing of Ha₂₅₅₋₂₆₂ with K^{k} through this double-chimeric β₂m (dcβ₂m ) was achieved in-spite of strong competition from cytosolic-derived K^{k}-restricted peptides. Although data cannot be directly compared, it is to be noted that high level of surface class I-bound peptide by expression of non-membrane-attached β₂m/peptide alone, could not be directly demonstrated in previous studies (Uger and Barber, 1998; Tafuro et al., 2001). Membranal anchorage of dcβ₂m is therefore likely to result in substantial augmentation in the overall density of desired class I antigenic peptides on the cell surface, thus offering a novel and unique tool for CTL induction.

The main objectives of the present invention are to develop both a cell based-vaccine and a DNA vaccine, based on membranal β₂m carrying at least one antigenic peptide covalently bound to its amino terminal, wherein said antigenic peptide is not a peptide related to an autoimmune disease.

As used herein, the terms “antigenic peptide” or “peptide or epitope derived from an antigen” mean both a peptide having a sequence comprised within the sequence of said antigen or an altered sequence, in which one or more amino acid residues have been replaced by different amino acid residues, which may bear higher affinity for the MHC class I molecule.

Thus, in one aspect, the present invention provides a polynucleotide comprising a sequence encoding a polypeptide that is capable of high level presentation of antigenic peptides on antigen-presenting cells, wherein the polypeptide comprises a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope, wherein said antigenic peptide is not related to an autoimmune disease.

In one embodiment, the polypeptide stretch at the β₂-microglobulin carboxyl terminal consists of a bridge peptide, which spans the whole distance to the cell membrane, said bridge peptide being linked to a sequence which can exert the required anchoring function. The bridge peptide has preferably about 10-15 amino acid residues, and more preferably, has a sequence comprised within the membrane-proximal sequence of a class I heavy chain HLA molecule. In a most preferred embodiment, this bridge peptide has 13 amino acid residues comprised within the extracellular membrane-proximal sequence of the class I heavy chain HLA-A2 molecule, and is the peptide of SEQ ID NO:1, of the sequence LRWEPSSQPTIPI.

In one embodiment, the anchoring sequence to which the bridge peptide is linked is the full or partial transmembrane and/or cytoplasmic domain of a molecule selected from the group consisting of: (i) a human MHC class I molecule consisting of an HLA-A, HLA-B or HLA-C molecule; (ii) a costimulatory B7.1, B7.2 or CD40 molecule; and (iii) a signal transduction element capable of activating T cells or antigen-presenting cells.

In one embodiment, the anchoring residue (i) above is a sequence consisting of the transmembrane and cytoplasmic domains from the MHC class I heavy chain HLA-A2 molecule, of the SEQ ID NO: 2, of the sequence:

VGIIAGLVLFGAVITGAWAAVMWRRKSSDRKGGSYSQAASSDSAQ GSDVSLTACKV

In another embodiment, the anchoring residue (iii) above is the intracellular region of a suitable signal transduction element capable of activating T cells such as, but not being limited to, a component of T-cell receptor CD3 such as the zeta (ζ) or eta (η) polypeptide, a B cell receptor polypeptide or an Fc receptor polypeptide. The cytoplasmic regions of the CD3 chains contain a motif designated the immunoreceptor tyrosine-based activation motif (ITAM), which has been shown to associate with cytoplasmic tyrosine kinases and to participate in signal transduction following TCR-mediated triggering. This motif is found in a number of other receptors including the Ig-α/Ig-β heterodimer of the B-cell receptor complex and Fc receptors for IgE and IgG, and three copies of it are found in the long cytoplasmic domains of the the ζ and η chains.

In a preferred embodiment, the anchoring residue of the chimeric molecule comprises the transmembranal and cytoplasmic regions of the human T-cell receptor CD3 ζ polypeptide, a signal transduction element capable of activating T cells.

In another embodiment, the signal transduction element capable of activating T cells comprises the transmembranal and cytoplasmic regions of a B-cell receptor polypeptide such as the Ig-α or Ig-β chain, the cytoplasmic tails in both being long enough to interact with intracellular signaling molecules. In a further embodiment, the signal transduction element comprises the transmembranal and cytoplasmic regions of Fc receptor polypeptides such as FcεRI, FcγRI or FcγRIII chains. FcεRI, a high-affinity receptor expressed on the surface of mast cells and basophils, contains four polypeptide chains: an α and a β chain and two identical disulfide-linked γ chains that extend a considerable distance into the cytoplasm and each has an ITAM motif. FcγRI, or CD64, is the high affinity receptor for IgG, expressed mainly on macrophages, neutrophils, eosinophils and dendritic cells. It comprises an α chain and two disulfide-linked α chains. This structure is also typical to FcγRIII, or CD16, which is the low affinity receptor for IgG, found on NK cells, eosinophils, macrophages, neutrophils and mast cells. CD3 ζ chain is found instead of the γ chain in a fraction of FcγRIII.

In still a further embodiment, the anchoring residue to which the bridge peptide is linked through its carboxyl terminal is a glycosylphosphatidylinositol (GPI)-anchor sequence, preferably the GPI-anchor peptide of SEQ ID NO:3, of the sequence FTLTGLLGTLVTMGLLT (from the protein DAF—complement decay-accelerating factor precursor or CD55 antigen; SWISSProt ID P08174, positions 365-381).

In one embodiment, the polynucleotide of the invention comprises a sequence encoding a polypeptide as defined in which the at least one non-autoimmune disease related antigenic peptide comprising a MHC class I epitope is linked to the β₂-microglobulin amino terminal directly. In another embodiment, the at least one antigenic peptide is linked to the β₂-microglobulin amino terminal through a peptide linker.

In one embodiment, the at least one antigenic peptide is at least one antigenic determinant of one sole antigen.

In another embodiment, the at least one antigenic peptide is at least one antigenic determinant of each one of at least two different antigens.

In one preferred embodiment of the invention, the at least one non-autoimmune disease related antigenic peptide comprising a MHC class I epitope linked to the β₂-microglobulin amino terminal is derived from a tumor-associated antigen (TAA) such as, but not limited to, alpha-fetoprotein, BA-46/lactadherin, BAGE (B antigen), BCR-ABL fusion protein, beta-catenin, CASP-8 (caspase-8), CDK4 (cyclin-dependent kinase 4), CEA (carcinoembryonic antigen), CRIPTO-1 (teratocarcinoma-derived growth factor), elongation factor 2, ETV6-AML1 fusion protein, G250/MN/CAIX, GAGE, gp100 gp100 (glycoprotein 100)/Pmel17, HER-2/neu (human epidermal receptor-2/neurological), intestinal carboxyl esterase, KIAA0205, MAGE (melanoma antigen), MART-1/Melan-A (melanoma antigen recognized by T cells/melanoma antigen A), MUC-1 (mucin 1), N-ras, p53, PAP (prostate acid phosphatase), PSA (prostate specific antigen), PSMA (prostate specific membrane antigen), telomerase, TRP-1/gp75 (tyrosinase related protein 1, or gp75), TRP-2, tyrosinase, and uroplakin Ia, Ib, II and III.

Examples of TAA peptides include, without being limited to, the following antigenic peptides:
(i) the HLA-A2 restricted human alpha-fetoprotein peptide GVALQTMKQ (SEQ ID NO:4 ) associated with liver tumors;
(ii) the HLA-Cw16 restricted human BAGE-1 peptide AARAVFLAL (SEQ ID NO:5);
(iii) the HLA-A2 restricted human BCR-ABL fusion protein (b3a2) peptide SSKALQRPV (SEQ ID NO:6) associated with chronic myeloid leukemia;
(iv) the HLA-A24 restricted human beta-catenin peptide SYLDSGIIF (SEQ ID NO:7) associated with melanoma;
(v) the HLA-A2 restricted human CDK4 peptide ACDPHSGHFV (SEQ ID NO:8) associated with melanoma;
(vi) the HLA-A2 restricted human CEA peptide YLSGANLNL (SEQ ID NO: 9) associated with gut carcinoma;
(vii) the HLA-A68 restricted human elongation factor 2 peptide ETVSEQSNV (SEQ ID NO:10) associated with lung squamous cell carcinoma;
(viii) the HLA-A2 restricted human ETV6-AML1 fusion protein peptide RIAECILGM (SEQ ID NO: 11) associated with acute lymphoblastic leukemia;
(ix) the HLA-A2 restricted human G250 peptide HLSTAFARV (SEQ ID NO:12) associated with stomach, liver and pancreas tumors;
(x) the HLA-Cw6 restricted human GAGE-1,2,8 peptide YRPRPRRY (SEQ ID NO:13);
(xi) the gp100 human peptides associated with melanoma HLA-A2 restricted KTWGQYWQV (SEQ ID NO:14), (A)MLGTHTMEV (SEQ ID NO:15), ITDQVPFSV (SEQ ID NO:16), YLEPGPVTA (SEQ ID NO:17), LLDGTATLRL (SEQ ID NO:18), VLYRYGSFSV (SEQ ID NO:19), SLADTNSLAV (SEQ ID NO:20 ), RLMKQDFSV

(SEQ ID NO:21), RLPRIFCSC (SEQ ID NO:22), and the HLA-A3 restricted LIYRRRLMK (SEQ ID NO:23), ALLAVGATK (SEQ ID NO:24), LALNFPGSQK (SEQ ID NO:25) and ALNFPGSQK (SEQ ID NO:26);
(xii) the HLA-A2 restricted human HER-2/neu ubiquitous peptide KIFGSLAFL (SEQ ID NO: 27);
(xiii) the HLA-B7 restricted human intestinal carboxyl esterase peptide SPRWWPTCL (SEQ ID NO:28) associated with liver, intestine and kidney tumors;
(xiv) the HLA-B44 restricted human KIAA0205 peptide AEPIMQTW (SEQ ID NO:29).associated with bladder tumor;
(xv) the MAGE-1 peptides HLA-A1 restricted human EADPTGHSY (SEQ ID NO:30) and HLA-A3 restricted human SLFRAVITK (SEQ ID NO:31);
(xvi) the MAGE-3 peptides HLA-A1 restricted human EVDPIGHLY (SEQ ID NO:32) and HLA-A2 restricted human FLWGPRALV (SEQ ID NO:33);
(xvii) the HLA-A2 restricted human MART-1/Melan-A peptide (E)AAGIGILTV (SEQ ID NO:34) associated with melanoma;
(xviii) the HLA-A2 restricted human MUC-1 peptide STAPPVHNV (SEQ ID NO:35) associated with glandular epithelia carcinoma;
(xix) the HLA-A1 restricted human N-ras peptide ILDTAGREEY (SEQ ID NO:36) associated with melanoma;
(xx) the HLA-A2 restricted human p53 ubiquitous peptide LLGRNSFEV (SEQ ID NO:37);
(xxi) the HLA-A2 restricted human PSA peptides FLTPKKLQCV (SEQ ID NO:38) and VISNDVCAQV (SEQ ID NO:39) associated with prostate carcinoma;
(xxii) the HLA-A2 restricted human telomerase peptide ILAKFLHWL (SEQ ID NO: 40) associated with testis, thymus, bone marrow, and lymph nodes carcinomas;
(xxiii) the HLA-A31 restricted human TRP-1 peptide MSLQRQFLR (SEQ ID NO:41) associated with melanoma;
(xxiv) the HLA-A2 restricted human TRP-2 peptides LLGPGRPYR (SEQ ID NO:42), SVYDFFVWL (SEQ ID NO:43), and TLDSQVMSL (SEQ ID NO:44) associated with melanoma;
(xxv) the HLA-A68 restricted human TRP2-INT2 peptide EVISCKLIKR (SEQ ID NO:45); and
(xxvi) the HLA-A1 restricted human tyrosinase peptide KCDICTDEY (SEQ ID NO:46) associated with melanoma.

This list is presented only as examples of TAA peptides that can be used according to the invention. However, it is intended to encompass within the scope any TAA peptide known or to be discovered in the future as periodically published in Cancer Immunity, a Journal of the Academy of Cancer Immunology, at the website http://www.cancerimmunity.org/peptidedatabase/Tcellepitopes.htm.

In one embodiment of the invention, the polynucleotide encodes a polypeptide comprising at least one antigenic determinant of one sole TAA. In another preferred embodiment, the polynucleotide encodes a polypeptide comprising at least one antigenic determinant of each one of at least two different TAAs.

Thus, in some applications according to the invention, it may be desired to link more than one epitope to the amino terminal of the anchored β₂m. In this way, the product of a single DNA molecule can mediate the induction of CTL clones directed at different epitopes from the same TAA, or from two or more different TAAs, restricted by one or more HLA class I allelic products.

In one embodiment, the two or more epitopes may be derived from the same antigen. For example, at least 9 different HLA-A2 binding peptides and 4 different HLA-A3 binding peptides derived from the melanoma-associated antigen gp100 have been identified. A melanoma patient, who carries both HLA-A2 and HLA-A3, can, in principle, mount CTL responses to these 13 different gp100-derived peptides.

Thus, in one preferred embodiment, the at least one antigenic peptide is at least one HLA-A2 binding peptide and at least one HLA-A3 binding peptide derived from the melanoma-associated antigen gp100, more preferably at least one gp100 HLA-A2 binding peptide selected from the group consisting of SEQ ID NO: 14, 15, 16, 17, 18, 19, 20, 21 and 22, and at least one gp100 HLA-A3 binding peptide selected from the group consisting of SEQ ID NO: 23, 24, 25 and 26.

In another embodiment, this strategy can be employed to elicit a CTL response to more than one antigenic molecule by using a single gene encoding epitopes of two different TAAs. For example, the same sequence can harbor peptides from gp100 and Melan-A/MART-1, both associated with melanoma, and harbor several HLA-A2-binding peptides, preferably at least one gp100 HLA-A2 binding peptide selected from the group consisting-of SEQ ID NO: 14, 15, 16, 17, 18, 19, 20, 21 and 22, and at least one Melan-A/MART-1 HLA-A2 binding peptide selected from the group consisting of SEQ ID NO: 34. Similarly, peptides from different antigens, which bind different class I alleles can be incorporated on the same construct, e.g., HLA-A3-restricted gp100 and HLA-A2-restricted Melan-A/MART-1 peptide(s). Similarly, other combinations of different TAAs related to melanoma can be formed using one or more of the melanoma-associated TAAs described above, e.g. peptides derived from beta-catenin, CDK4, gp100, Melan-A/MART-1, N-ras, TRP-1, TRP-2, and tyrosinase.

In another preferred embodiment of the invention, the at least one non-autoimmune disease related antigenic peptide comprising a MHC class I epitope linked to the β₂-Microglobulin amino terminal is derived from an antigen from a pathogen selected from the group consisting of a bacterial, a viral, a fungal and a parasite antigen.

Examples of antigens derived from pathogenic, e.g. infectious, agents are, without being limited to, antigens derived from an organism selected from the group comprising: human immunodeficiency virus HIV (Takahashi et al., 1993), varicella zoster virus, herpes simplex virus type 1 (HSV-1), herpes simplex virus type 2 (HSV-2), human cytomegalovirus (CMV), dengue virus, hepatitis A, B, C or E, respiratory syncytial virus, human papilloma virus, influenza virus, Hib, meningitis virus, Salmonella, Neisseria, Borrelia, Chlamydia, Bordetella, Streptococcus, Mycoplasma, Mycobacteria, Haemophilus, Plasmodium or Toxoplasma, stanworth decapeptide; and TCR idiotypic peptides shared by autoreactive T cells (Cohen and Weiner, 1998; Offner et al., 1999; Kumar and Sercarz, 1999).

In one preferred embodiment, the pathogen antigen is a viral antigen such as, but not limited to, hepatitis virus, cytomegalovirus, or HIV viral antigen consisting of an HIV protein selected from the group consisting of the HIV-1 regulatory proteins Tat and Rev and the HV envelope protein, in which case the antigenic peptide derived therefrom has the sequence RGPGRAFVTI (SEQ ID NO: 47).

In one embodiment of the invention, the polynucleotide encodes a polypeptide comprising at least one antigenic determinant of one sole pathogen antigen. In another preferred embodiment, the polynucleotide encodes a polypeptide comprising at least one antigenic determinant of each one of at least two different pathogen antigens. In this way, the product of a single DNA molecule can mediate the induction of CTL clones directed at different epitopes from the same viral antigen or from two or more different viral antigens, restricted by one or more HLA class I allelic products. For example, against AIDS, a combination of epitopes derived from each of the Tat, Rev and the HIV envelope proteins, may be used.

In yet a further embodiment of the invention, the at least one non-autoimmune disease related antigenic peptide comprising a NMHC class I epitope linked to the β₂-microglobulin amino terminal is at least one idiotypic peptide expressed by autoreactive T lymphocytes. The idiotypic peptide is preferably derived from a CDR (complementarity-determining region), more preferably CDR3, of an immunoglobulin or of a TCR chain, and it may also contain CDR flanking segments.

This embodiment is suitable for some applications according to the invention that may require the covalent linking of longer polypeptide stretches, which may contain one or more epitopes of unknown class I binding properties. For example, idiotypic peptides derived from CDRs (especially CDR3) of immunoglobulin or TCR polypeptide chains can be employed for the induction of CTL response against lymphomas and leukemias of both B cell and T cell origin (Wen and Lim, 1993; Berger et al., 1998) or against autoreactive T cell clones (Kumar et al., 1995). However, many of these sequences are clonotypic in nature and there are no preliminary data concerning class I binding capacity of peptides they comprise. In such cases, longer DNA inserts, encoding, for example, not only the relevant CDR3 sequence, but also parts of its flanking FR3 and FR4 segments can be cloned directly from tumor cells or autoreactive T cell clones associated with an autoimmune disease. If the encoded stretch contains one or more peptides which can bind one or more of the patient's HLA class I products, the obtained dcβ₂m will induce CTLs of the corresponding specificities.

This task can be accomplished by the genetic insertion of the fragment encoding the longer peptide into the expression vector between the sequence encoding the leader peptide (the leader peptide or signal peptide is the peptide stretch at the amino terminal of any newly synthesized polypeptide chain, which is to be translocated to the ER) and the sequence coding for the linker peptide. The fragment encoding the longer peptide can be prepared with the use of synthetic oligonucleotides or as a PCR product (as for the CDR3 idiotypic peptides, using sets of FR3- and FR4-specific primers), or by any other procedure commonly used for molecular cloning. This design is based on the observations that MHC class I molecules can accommodate longer peptides than the canonical size of 8-10 amino acids. This most likely occurs by protrusion rather than by bulging (Stryhn et al., 2000) and shows preference to carboxyl terminal rather than to amino terminal extensions (Horig et al., 1999). It is predicted that in each assembly event in the ER of a relevant MHC class I molecule, a different peptide from the same dcβ₂m gene product can associate with the nascent MHC class I heavy chain. Following this association, the amino terminal protrusion can be trimmed by an ER aminopeptidase, operative in the early secretory pathway, as suggested by Snyder et al., 1994, and recently identified as the ER aminopeptidase ERAAP (Serwold et al., 2002) or ERAP1 (York et al., 2002; Saric et al., 2002), which trims precursors to MHC class 1-presented peptides. The mature class I molecule will then be ready for transportation to the cell membrane. The rest of the long peptide may still link through its carboxyl terminal to the membranal β₂m. Hence, enhanced complex stability and, concomitantly, high level of presentation are expected. In this manner, a panel of ligands can be formed in the APCs for induction of CTLs with different specificities, as the result of delivery of a single gene. This prediction also pertains to idiotypic peptides: an epitope can be embedded anywhere along the cloned sequence, and, similarly, the amino terminal protrusion will be cleaved. It is highly likely that there will be a functional limitation to the size of the linked stretch, and that secondary structures formed within this stretch will interfere with the ability of at least some of the embedded epitopes to be properly presented.

In a more preferred embodiment of the invention, the polynucleotide of the invention as described hereinbefore is an expression vector and comprises a vector and regulatory sequences along with the polynucleotide sequence.

In another aspect, the present invention provides an expression vector comprising a polynucleotide of the invention as described hereinbefore.

Any suitable mammalian expression vector can be used such as, but not limited to, the pCI mammalian expression vectors (Promega, Madison, Wis., USA), pCDNA3 expression vectors (Invitrogen, San Diego, Calif.) and pBJ1-Neo. The expression vector may also be a plasmid DNA in which the polynucleotide sequence is controlled by a virus, e.g. cytomegalovirus, promoter, or, most preferably, the expression vector is a recombinant viral vector such as, but not limited to, pox virus or adenovirus or adeno-associated viral vector.

In a further aspect, the present invention provides an antigen-presenting cell (APC) transfected with a polynucleotide comprising a sequence encoding a dcβ₂m of the invention, i.e. a polypeptide comprising a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope.

The APC may be a macrophage, a B cell, a fibroblast and, more preferably, a dendritic cell. In a preferred embodiment, the antigenic peptide is a peptide not related to an autoimmune disease.

In one embodiment, the at least one antigenic peptide in the antigen-presenting cell is at least one peptide derived from at least one TAA. Said cell is capable of presenting the at least one TAA peptide at a sufficiently high density to allow potent activation of peptide-specific cytotoxic T lymphocytes (CTL) capable of recognizing and binding to harmful tumor cells and causing their elimination or inactivation.

In another embodiment, the at least one antigenic peptide in the antigen-presenting cell is at least one peptide derived from an antigen from a pathogen selected from the group consisting of a bacterial, a viral, a fungal and a parasite antigen.

In another embodiment, the at least one antigenic peptide in the antigen-presenting cell is at least one idiotypic peptide expressed by autoreactive T lymphocytes, preferably at least one idiotypic peptide derived from a CDR, more preferably CDR3, of an immunoglobulin or of a TCR chain, that may also contain CDR flanking segments.

Any of the techniques which are available in the art may be used to introduce the recombinant nucleic acid encoding the polypeptide into the antigen presenting cell. These techniques are collectively referred to as transfection herein and include, but are not limited to, transfection with naked or encapsulated nucleic acids, cellular fusion, protoplast fusion, viral infection, cellular endocytosis of calcium-nucleic acid microprecipitates, fusion with liposomes containing nucleic acids, and electroporation. Choice of suitable vectors for expression is well within the skill of the art. Antigen expression may be determined by any of a variety of methods known in the art, such as immunocytochemistry, ELISA, Western blotting, radioimmunoassay, or protein fingerprinting.

In an additional aspect of the present invention, a DNA vaccine is provided comprising a polynucleotide of the invention or an expression vector of the invention, both as described hereinabove.

In one embodiment, there is provided a DNA vaccine for prevention or treatment of cancer comprising a polynucleotide that encodes a polypeptide comprising at least one antigenic determinant of at least one TAA.

In another embodiment, there is provided a DNA vaccine for prevention or treatment of a disease caused by a pathogenic organism comprising a polynucleotide that encodes a polypeptide comprising at least one antigenic determinant of at least one pathogenic antigen.

The DNA vaccines may be constructed according to methods known in the art. Genes in plasmid expression vectors are expressed in vivo after intramuscular (i.m.) or subcutaneous (s.c.) injection and this expression stimulates an immune response against the plasmid-encoded proteins. The same or better effect is obtained replacing the plasmid by a viral vector.

In one embodiment, the DNA vaccine is a naked DNA vaccine. It may contain a plasmid DNA that contains the polynucleotide of the invention controlled by a cytomegalovirus (CMV) promoter. When the plasmid is introduced into mammalian cells, cell machinery transcribes and translates the gene. The expressed protein (immunogen) is then presented to the immune system where it can elicit an immune response. One method of introducing DNA into cells is by using a gene gun. This method of vaccination involves using pressurized helium gas to accelerate DNA-coated gold beads into the skin of the vaccinee.

DNA vaccines are capable of eliciting both strong humoral and cell-mediated immunity. Therefore DNA immunization represents a new approach for prevention (vaccination) and treatment (immune-based therapy) of infectious and neoplastic diseases.

In yet a further aspect of the invention, there is provided a cellular vaccine which comprises an antigen presenting cell of the invention as described hereinbefore. The antigen presenting cell is preferably a dendritic cell, but may also be a macrophage, a B cell and a fibroblast. The cells in the cellular vaccine may be autologous, allogeneic or xenogeneic cells.

The present invention provides cellular vaccines which comprise an antigen presenting cell that is capable of presenting at least one antigenic peptide comprising an epitope of at least one antigen and has the ability to induce potent CTL responses against the desired antigen(s). Vaccination, as used herein, refers to the step of administering the cellular vaccine to a mammal to induce such an immune response, for example, to prevent or treat a tumor or a disease caused by an infectious agent in a mammal.

The presentation of the at least one antigenic peptide by the APCs in the cellular vaccine can be achieved by transfecting the APCs with the polynucleotide of the invention, or by transducing the APCs with a virus encoding the polynucleotide of the invention or by incubating said antigen presenting cells with a polynucleotide encoding said at least one antigenic peptide.

In one embodiment, the invention provides a cellular vaccine for prevention or treatment of cancer wherein the antigen presenting cell presents at least one peptide derived from at least one tumor associated antigen.

In an additional aspect, the present invention provides a cellular vaccine for the prevention and/or treatment of a cancer comprising antigen presenting cells which express a scβ₂m, i.e. a β₂-microglobulin linked through its carboxyl terminal to a polypeptide stretch that allows the anchorage of the β₂-microglobulin molecule to a cell membrane, wherein said polypeptide stretch consists of a bridge peptide which spans the whole distance to the cell membrane, said bridge peptide being linked to a sequence which can exert the required anchoring function, and wherein said cells have been pulsed with at least one antigenic peptide derived from at least one tumor associated antigen.

In still a further aspect, for the treatment of cancer it is envisaged by the present invention to encompass tumor cells transfected with a polynucleotide comprising a sequence encoding a scβ₂m, i.e. a polypeptide comprising a β₂-microglobulin molecule that is linked through its carboxyl terminal to a polypeptide stretch that allows the anchorage of the β₂-microglobulin molecule to the cell membrane. The scβ₂m will enhance expression of the MHC class 1 molecules on the cell surface of the tumor cells.

In this aspect, it is known that tumor cells, which manifest impaired expression of MHC class I MHC molecules and are thus poorly immunogenic, can induce antitumor CTL activity upon transfection of MHC class I genes (Feldman and Eisenbach, 1991). The level of MHC class I expressed on the surface of tumor cells is a key factor, which governs immunogenicity of the tumor, and is amenable to genetic modification. It is evident from Table 2 hereinafter that the mere expression of scβ₂m results in 3-4-fold enhancement in the level of H-2K^{k}. This effect can be harnessed to augment MHC class I expression by tumor cells. For example, tumor cells can be derived from the patient, transduced ex-vivo with a recombinant virus encoding membranal hβ₂m and expanded. Following their mitotic inactivation, transduced cells will be introduced back to the patient to serve as immunogens capable of eliciting a tumor-specific CTL response. This response may then target also unmodified tumor cells, provided they still express MHC class I molecules at a level sufficient for recognition by the armed effector CTLs.

In another embodiment, the invention provides a cellular vaccine for prevention or treatment of a disease caused by a pathogenic organism wherein the antigen presenting cell presents at least one peptide derived from a pathogenic antigen.

In a further additional aspect, the present invention provides a cellular vaccine for the prevention and/or treatment of a disease caused by a pathogen comprising antigen presenting cells which express a scβ₂m, i.e. a β₂-microglobulin linked through its carboxyl terminal to a polypeptide stretch that allows the anchorage of the β₂-microglobulin molecule to a cell membrane, wherein said polypeptide stretch consists of a bridge peptide which spans the whole distance to the cell membrane, said bridge peptide being linked to a sequence which can exert the required anchoring function, and wherein said cells have been pulsed with at least one antigenic peptide derived from at least one antigen of said pathogen.

The cellular vaccine may be administered subcutaneously, intradermally, intratracheally, intranasally, or intravenously. The cells may be suspended in any pharmaceutically acceptable carrier, such as saline or phosphate-buffered saline.

In still another aspect, the present invention provides a method of immunizing a mammal against a tumor-associated antigen comprising the step of: immunizing the mammal with an antigen presenting cell which has been transfected with, or transduced with, or loaded with, a recombinant nucleic acid molecule comprising a sequence encoding a polypeptide comprising a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope of at least one tumor-associated antigen, or with a cellular vaccine comprising said antigen presenting cell, wherein the mammal mounts a cytotoxic immune response against the at least one tumor-associated antigen, and wherein the antigen presenting cell presents said at least one antigenic peptide.

In yet another aspect, the present invention provides a method of immunizing a mammal against a disease caused by a pathogenic organism comprising the step of: immunizing the mammal with an antigen presenting cell which has been transfected with, or loaded with, a recombinant nucleic acid molecule comprising a sequence encoding a polypeptide comprising a β₂-microglobulin molecule that is linked through its carboxyl terminal to a polypeptide stretch that allows the anchorage of the β₂-microglobulin molecule to the cell membrane, and through its amino terminal to at least one antigenic peptide comprising a MHC class I epitope of a pathogenic antigen, or with a cellular vaccine comprising said antigen presenting cell, wherein the mammal mounts a cytotoxic immune response against the pathogenic antigen, and wherein the antigen presenting cell presents said at least one antigenic peptide.

In still a further aspect, the present invention provides a method for the prevention and/or treatment of a cancer or of a disease caused by a pathogen which comprises administering to a patient in need thereof antigen presenting cells which express a chimeric polypeptide comprising β₂-Microglobulin linked through its carboxyl terminal to a polypeptide stretch that allows the anchorage of the β₂-microglobulin molecule to a cell membrane, wherein said polypeptide stretch consists of a bridge peptide which spans the whole distance to the cell membrane, said bridge peptide being linked to a sequence which can exert the required anchoring function, and wherein at least one antigenic peptide derived from at least one tumor associated antigen or from an antigen of said pathogen is exogenously loaded on said antigen presenting cells, preferably in the grooves of the MHC complex formed by the association of the chimeric polypeptide with the endogenous MHC molecule component.

In yet still a further aspect, the present invention provides pharmaceutical compositions. In one embodiment, the composition comprises as an active ingredient at least one polynucleotide or an expression vector of the invention, and a pharmaceutically acceptable carrier. The polynucleotide may comprise a sequence encoding a polypeptide comprising at least one antigenic peptide derived from at least one tumor associated antigen, or at least one antigenic peptide derived from a pathogenic antigen. In another embodiment, the pharmaceutical composition comprises as an active ingredient at least one antigen presenting cell of the invention, and a pharmaceutically acceptable carrier.

Good cancer vaccines should induce a protective CTL response directed at MHC class I peptides derived from TAAs. The pivotal APC in CTL priming is the dendritic cell (DC), which has indeed been widely utilized in the design of cancer vaccines. In particular, DCs are attributed a critical role in DNA immunization, and direct presentation of peptides derived from expression of genetic material internalized by DCs is considered a major route for CTL induction. While magnitude of a CTL response correlates with density of specific MHC-peptide complexes on the APC surface, many TAA peptides have low affinity for the class I molecule and are presented at sub-optimal densities. Combined with the limiting expression level normally achieved following administration of non-replicating DNA, DNA immunization against TAAs usually falls short from achieving the anticipated effect.

The double-chimeric β₂m (dcβ₂m) polypeptide design of the present invention creates an entirely novel MHC class I entity, which may offer a great advantage over current strategies as a means to augment CTL induction. The membrane anchorage of the β₂m molecule can be achieved by covalently linking to its carboxyl terminal a peptide bridge, which spans the whole distance to the cell membrane, and is supplemented by an anchoring sequence such as the transmembrane and cytoplasmic domains derived from another cell surface protein. Following dissociation of β₂m-linked peptide from the α chain, this design is expected to prevent detachment of the β₂m/peptide from the cell membrane. Membrane anchorage should immensely increase the local concentration of dcβ₂m in the cell membrane, and allow rapid re-formation or de-novo formation of the specific MHC class I complex upon peptide dissociation. This will significantly prolong the actual half-life of the complex, and increase its membranal level.

Chimeric β₂m polypeptides having a sole antigenic peptide linked to their amino terminal, which are provided exogenously, have been shown to associate with α chains on the cell surface and to form fall MHC class I complexes (Uger and Barber, 1998' Tafuro et al., 2001; Uger et al., 1999; White et al., 1999). According to the present invention, it is also assumed that re-association will take place on the cell membrane but obeying kinetics of lateral diffusion. Furthermore, but not less important, the high local peptide concentration, the membranal form of β₂m and the anticipated proteasome- and TAP-independence according to the invention, are all expected to render initial assembly of the specific, intact MHC class I complex in the ER highly favorable, compared with assembly involving processing and transportation of conventional, cytosolic peptides.

As used herein, the term “double-chimeric β₂-Microglobulin” (dcβ₂m) refers to a molecule of β₂m having at least one epitope/antigenic peptide bound to the amino terminal and an anchor domain bound to the carboxyl terminal, wherein said anchor domain is composed of a polypeptide stretch consisting of a bridge peptide, which spans the whole distance to the cell membrane, and a peptide sequence that allows the anchorage of the β₂-microglobulin molecule to the cell membrane. The term “single-chimeric β₂-microglobulin” (scβ₂m), when used herein, refers to a molecule of β₂m having only the anchor domain, as defined above, but no antigenic peptide at the amino terminal.

The realization that vaccination with naked DNA results in long-lasting protein expression and stimulation of specific humoral and cellular immune responses, has made a large impact in the field of vaccine design (see Gurunathan et al., 2000 for review). Numerous studies, which have shown that DNA vaccines induce potent MHC class I-restricted CTL responses against TAAs, have suggested that this modality may be particularly useful for the treatment of cancer, and have prompted the development of a variety of DNA vaccine strategies (see review by Benton and Kennedy, 1998). First human trials of cancer DNA vaccines have been initiated, but it is too early to evaluate their efficacy. There is compelling evidence that a CTL response following DNA administration can be induced by directly transfected DCs (Porgador et al., 1998), although other mechanisms, such as direct transfection of somatic cells or cross presentation by DCs, are also considered.

According to the present invention, direct delivery of the dcβ₂m polypeptide produced by DCs, which express the introduced gene, to surface MHC class I molecules for peptide presentation is expected to result in considerable enhancement in peptide level, and hence, in vaccine efficacy, compared with that achieved by conventional antigen processing and presentation.

The present invention thus provides a novel and broadly-applicable strategy for efficient induction of antigen-specific CTLs, which is based on the ability of dcβ₂m to markedly enhance presentation of antigenic peptides. The CTL response may be optimized by a regimen of two or more booster administrations. Cocktails of two or more CTL inducing peptides are employed to optimize epitope and/or MHC class I restricted coverage.

For the purposes of the present invention, the biochemical and immunological properties associated with this mode of presentation are first explored in vitro in transfected cell lines, and its in vivo function is then assessed in a mouse melanoma tumor model, applying transfected APC cell lines, naked DNA immunization and adoptive transfer of syngeneic APCs from transgenic mice.

Defining various parameters, which govern expression of dcβ₂m, and establishing its actual potential as a tumor vaccine in a mouse model are expected to pave the way for the design of a novel modality of human cancer vaccines. The most suitable effector cells for this purpose are autologous DCs, which can be relatively easily transduced to express foreign genes (Hadzantonis and O'Neill, 1999; Bubenik, 2001).

The inability to present low affinity peptides at densities required for potent activation of the entire repertoire of peptide-specific CTL clones is considered a major obstacle in many of the current protocols, which aim at producing DC-based cancer vaccines. According to the present invention, it is expected that the dcβ₂m-based constructs will increase the apparent affinity of the peptide to the MHC molecule and, thus, the dcβ₂m-mediated presentation on DCs should allow TAA-derived peptides with limiting affinity for the restricting MHC class I product to be presented by the DCs at sufficiently high density. This is one of the expected advantages of the present invention in comparison to previously proposed approaches for the development of cancer vaccines based on dendritic cells.

Some TAAs are expected to play an active part in the induction of central tolerance in the thymus, thus allowing only CTLs of low avidity to mature (Gilboa, 1999). These may include TAAs which are classified as differentiation antigens (for example MART-1/Melan A, gp100 and tyrosinase), and, probably to a lesser extent, normal gene products with highly restricted tissue distribution (such as MAGE, BAGE and GAGE). The strategy of the present invention can be efficient in activating such low avidity CTLs.

Tumors often evade the immune system by reduction in MHC class I peptide presentation to CTLs by downregulation of either components of the proteasome complex or TAP (for review see Benton and Kennedy, 1998). Enhancement of TAA peptide presentation by such tumors following gene delivery activates CTLs, which can respond also to non-modified tumor cells (Sherritt et al., 2001), provided the density of class I tumor-associated epitopes exceeds a functional threshold of these CTLs. Hence, dcβ₂m or scβ₂m are expected to induce CTLs not only in professional APCs as dendritic cells but, in certain cases, also when expressed in tumor cells.

The approach of the proposed invention offers broad applicability and requires only straightforward genetic engineering: since human β₂m is monomorphic, only one cloning expression cassette should be prepared, to which the segment encoding any antigenic peptide of interest can easily be inserted.

The invention will now be illustrated by the following non-limiting examples.

### EXAMPLES

Materials and Methods

(i) Cells. MD45 is an H-2D^{b}-allospecific mouse H-2^{k/d} CTL hybridoma of BALB/c origin (Faufmann et al., 1981). RMA-S is a mutant cell line derived from the C57BL/6 lymphoma RMA (H-2^{b}), which has defects in peptide presentation by class I MHC molecules due to loss of functional expression of the TAP component TAP-2. These cells can be loaded exogenously with high levels of MHC class I compatible peptides. RMA/OVA and RMA-S/OVA are clones of these two cells transfected with the full-length chicken ovalbumin gene. B3Z is an H-2K^{b}-restricted, OVA₂₅₇₋₂₆₄-specific CTL hybridoma, harboring the NFAT-LacZ reporter gene (Sanderson and Shastri, 1994), and is a gift from Dr. N. Shastri, University of California, Berkeley. Three clones of the mouse melanoma B16, a spontaneously-arising melanoma of C57BL/6 origin are used: F10.9 is a spontaneously metastasizing clone of the B16-F10 line, K1 is an H-2K^{b} transfectant of F10.9 (orgador et al., 1989) and MO5 is a chicken ovalbumin-ransfected variant of the B16 melanoma. C57BL/6-derived T cell Line A, reactive with TRP-2 peptide 181-188 (TRP-2₁₈₁₋₁₈₈) (Bloom et al., 1997), is available from Dr. J. Yang, NCI, NIH, USA.

(ii) Antibodies. Fab13.4.1 is a Fab fragment specific to Ha₂₅₅₋₂₆₂ in the context of K^{k}, and was a gift from Dr. J. Engberg, University of Copenhagen (Andersen et al., 1996). AF3-12.1 is an anti-K^{k} mAb (Pharmingen). BM-63 is an anti-human β₂m mAb (Sigma). 20.8.4 is an anti-H-2K^{b} mAb. 28-14-8 is an anti-H-2Db mAb. 25-D1.16 is specific to the complex H-2K^{b}/OVA₂₅₇₋₂₆₄ (Porgador et al., 1997). These latter antibodies are available from Dr. L. Eisenbach, Weizmann Institute of Science, Rehovot, Israel.

(iii) DNA transfection. 5-10×10⁶ RMA-S cells in 0.8 ml were mixed in 4 mm sterile electroporation cuvette (ECU-104, EquiBio, Ashford, UK) with 10-20 μg DNA of the constructed plasmid and placed on ice. Transfection was performed by electroporation using Easyject Plus electroporation unit (EquiBio, Ashford, UK) at 350V, 750 μF. Cells were resuspended in fresh medium and cultured for 24-48 hours in 96-well plates prior to addition of the selecting drug (1 mg/ml G418). Resistant clones were first expanded in 24-well plates and analyzed for expression of the introduced gene by FACS.

(iv) FACS analysis. Cells were stained with indicated antibodies according to standard procedures and were subjected to flow cytometry analysis. 10⁶ cells were washed with phosphate-buffered saline (PBS) containing 0.02% sodium azide and incubated for 30 minutes on ice with 100 μl of the anti-human β₂m mAb (Sigma) at 10 μg/ml or the same concentration of a control antibody (or no antibody). Cells were then washed and incubated on ice with 100 μl of 1:100 dilution of goat anti-mouse IgG (FAB specific)-FITC conjugated polyclonal antibody (Sigma) for 30 minutes. Cells were washed and resuspended in PBS and analyzed by a FACSCalibur (BD Biosciences, Mountain View, Calif.). Statistical analysis was performed with the FACSCalibur CellQuest software. Quantitative analysis of cell surface antigens was performed with QIFIKIT (DAKO, Carpinteria, Calif.) according to the manufacturer's instructions.

(v) Cell stimulation assay. Cells at 5×10⁵ cells/ml were incubated overnight in 96-well plates in the presence of 5 μg/ml antibody (immobilized overnight and washed 3 times in PBS) or with target cells at 5×10⁵ cells/ml. Total volume: 0.1 ml.

(vi) In-cell X-Gal staining. Cells in 96-well plates were washed twice with PBS and fixed with 0.25% glutaraldehyde for 15 min, washed 3 times in PBS, incubated for 4 hours with 100 μl of X-Gal solution {0.2% X-Gal, 2mM MgCl₂, 5 mM K₄Fe(CN)₆3H2O, 5 mM K₃Fe(CN)₆ in PBS} and scored under the microscope for blue staining.

(vii) Immunization of mice. Immunization was carried out with peptide loaded RMA-S cells, RMA-S, RMA-S/OVA and RMA/OVA and OVA₂₅₇₋₂₆₄-dcβ₂m-expressing RMA-S transfectants (Y314-7 and Y317-2): RMA-S cells were incubated at 2×10⁶ cells/ml for 2 hours with 200 μg/ml of OVA₂₅₇₋₂₆₄. Mice were immunized twice i.p. with 2×10⁶ irradiated (50 Gy) cells, at 10 day intervals.

(viii) Cytotoxicity assay. Ten days after last immunization spleens were removed and single cell suspension were prepared. Splenocytes were restimulated with irradiated, mitomycin-C-treated tumor cells or target cells. Restimulated lymphocytes were maintained for another 4 days. Viable lymphocytes were separated on Lympholyte-M gradient (Cendarlane, Ontario, Canada) and resuspended at 5×10⁶/ml with lymphocyte medium. Lymphocytes were mixed at different ratios (1:100, 1:50, 1:25 and 1:12.5 target to effector) with ³⁵S-methionine-labeled target cells (tumor cells or peptide-presenting cells). CTL assays were carried out following standard procedures.

### Example 1

### Expression of dcβ₂m Designed for T cell Re-Programming

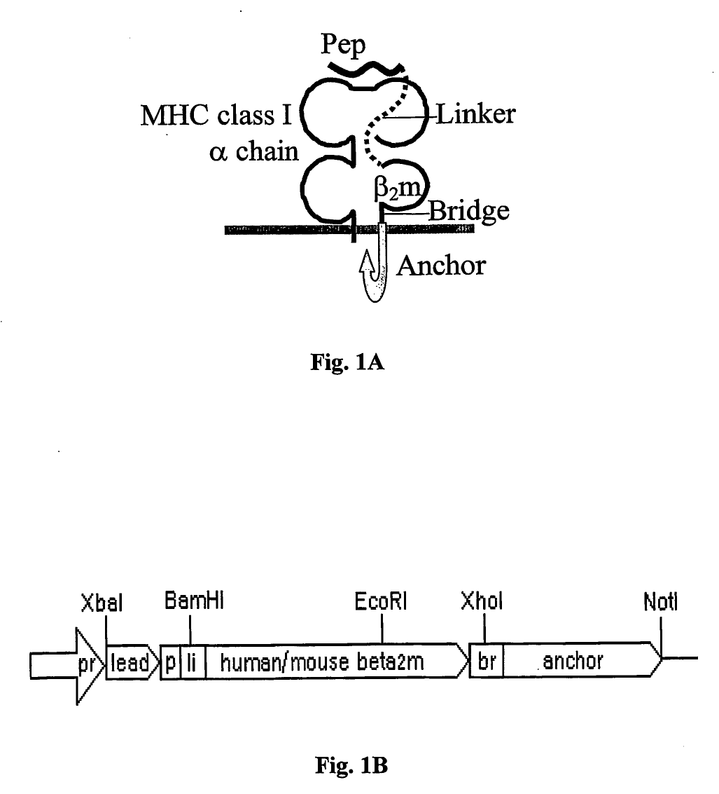

The general schemes of genetic constructs encoding dcβ₂m and of the polypeptide product associated with an MHC class I heavy chain are illustrated in FIG. 1. Table 1 summarizes all different single and double chimeric β₂m expression plasmids generated in this system as well in the tumor experimental system, which will be described below.

TABLE 1
Double and single chimeric β₂m constructs and
transfected clones expressing them

| Peptide | Allele | β₂2m | Anchor | Cell (H-2) | Clone |
|---|---|---|---|---|---|
| IV Ha₂₅₅₋₂₆₂ | K^{k} | human | none | MD45(k/d) | 840-7 |
| IV Ha₂₅₅₋₂₆₂ | K^{k} | human | CD3 ζ | MD45 | 427-24 |
| IV NP₅₀₋₅₇ | K^{k} | human | CD3 ζ | MD45 | 425-44 |
| Insulin B₁₅₋₂₃ | K^{d} | mouse | CD3 ζ | MD45 | 829S-36 |
| OVA₂₅₇₋₂₆₄ | K^{b} | mouse | H-2K^{b} | RMA-S(b) | Y314-7 |
| OVA₂₅₇₋₂₆₄ | K^{b} | human | H-2K^{b} | RMA-S | Y317-2 |
| TRP-2₁₈₁₋₁₈₈ | K^{b} | mouse | H-2K^{b} | RMA-S | Y313-10 |
| TRP-2₁₈₁₋₁₈₈ | K^{b} | human | H-2K^{b} | RMA-S | Y318-7 |
| none | — | human | CD3 ζ | RMA-S | KD21-4, 6 |
| none | — | human | H-2K^{b} | RMA-S | D323-4 |
| none | — | human | CD40 | B3Z(b) | Y340-13 |

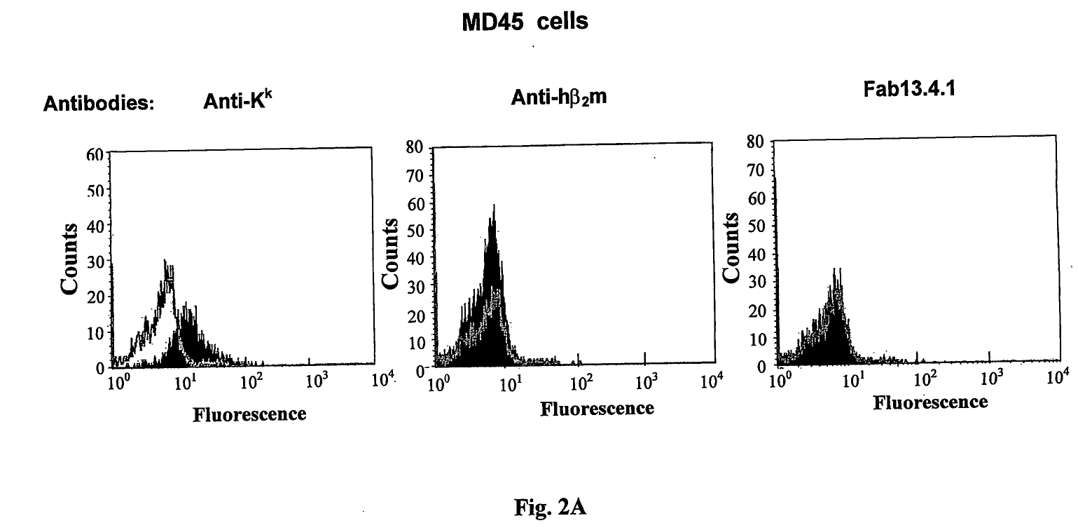

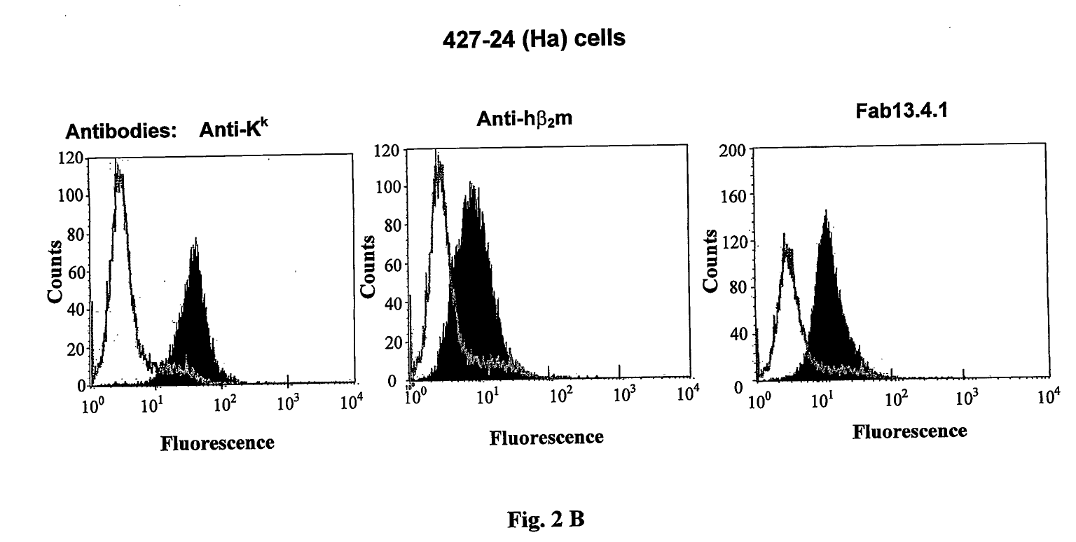

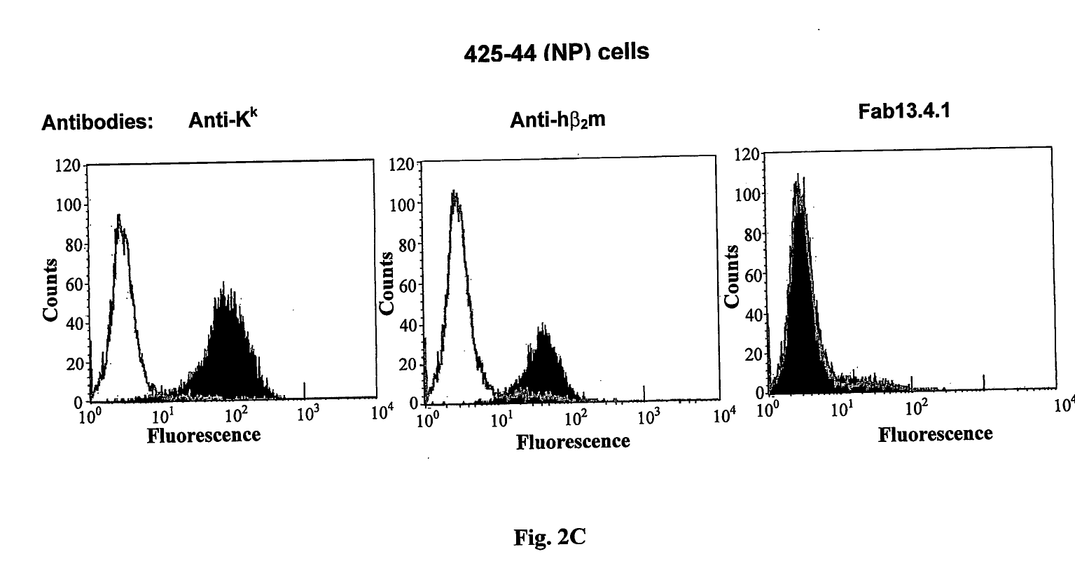

In our previous patent application, WO 01/91698, herein incorporated by reference as if fully disclosed herein, it was aimed to redirect effector T cells against other, harmful T cells, through the CD3 ζ chain portion. In the experimental system described in WO 01/91698, two special mammalian expression cassettes were constructed, which allow the single-step insertion of a stretch coding for an antigenic peptide, so as to create dcβ₂m of either human or mouse origin. The bridging peptide, derived from the human MHC class I molecule HLA-A2, was the extracellular 13-amino acid stretch of SEQ ID NO:1, which is most proximal to the cell membrane, and the transmembrane and cytoplasmic domains were those of the mouse CD3 ζ chain. The sequence encoding the K^{k}-restricted influenza virus hemagglutinin peptide Ha₂₅₅₋₂₆₂ was cloned into the unique cloning sites in the human β₂m cassette. Plasmid DNA was transfected into the MD45 hybridoma, and one stable transfectant, designated 427-24 (Ha), was further analyzed. Another MD45 transfectant, designated 425-44 (NP), was generated, which similarly expresses the K^{k}-restricted influenza virus nucleoprotein peptide NP₅₀₋₅₇. FACS analysis was performed with the anti-hβ₂m and anti-H-2K^{k} antibodies and with the K^{k}/Ha₂₅₅₋₂₆₂ complex-specific Fab13.4.1. FIG. 2 shows intensive staining of 427-24, but no detectable staining of the control cell 425-44 or of the parental MD45. Quantitative analysis of antigen level on the surface of both transfectants and parental MD45 cells is shown in Table 2 and reveals occupation of 20% of surface H-K^{k} molecules of 427-24 cells by the Ha₂₅₅₋₂₆₂ peptide.

TABLE 2
Quantitative analysis of surface antigens of transfectants 425-44
and 427-24 and parental MD45 cells*

| | Antibody | | |
|---|---|---|---|
| Cell | Anti-H-2K^{k} | Anti-hβ2m | Fab 13.4.1 |
| MD45 | 10,909 | 0 | 0 |
| 425-44 | 37,604 | 466,704 | 0 |
| 427-24 | 28,637 | 173,143 | 5,715 |
| *Cells were stained with the anti-H-2K^{k} mAb AF3-12.1, the anti-hβ2m mAb BM-63 and Fab 13.4.1, specific to the K^{k}/Ha₂₅₅₋₂₆₂ complex, and analyzed with QIFIKIT (Dako), using goat anti-mouse IgG (Fab-specific)-FITC conjugated polyclonal | | | |
| # antibodies. Mean fluorescence intensities were derived with FACSCalibur software and standard curve was generated from the linear regression of five points at 3,600, | | | |
| # 16,000, 53,000, 218,000 and 620,000 mouse IgG molecules per bead, using Excel. | | | |

It should be noted that the complex-specific antibody (Fab13.4.1) is a Fab, whereas the anti-H-2K^{k} is an intact IgG. Therefore, the actual occupation of H-2K^{k} molecules on the surface of 427-24 may in fact be higher. Also noteworthy is the 3-fold increase in the total amount of H-2K^{k} in both transfectants 425-44 and 427-24, compared with the parental MD45 cells.

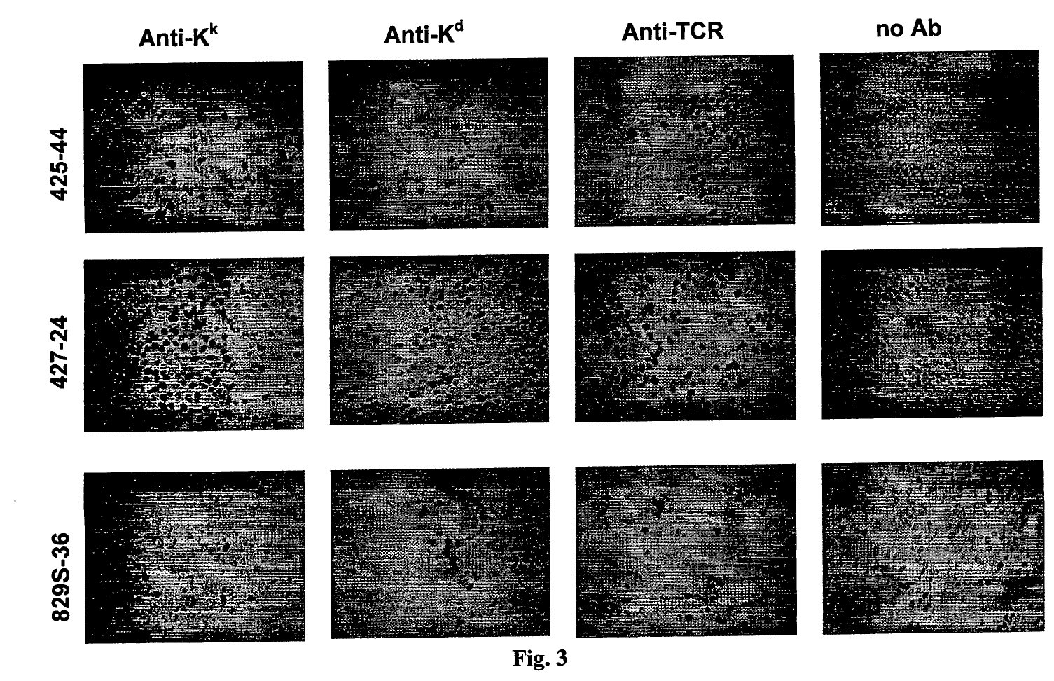

It is conceivable that, on the cell surface, dcβ₂m polypeptides can associate with MHC class I allelic products other than the restricting one. In this scenario, the flexible peptide linker allows the covalently linked antigenic peptide to be situated away from the MHC binding groove, which is occupied by a conventional peptide. In order to test this structural prediction we designed a functional assay, based on the ability of our transfectants to respond to stimulation by Lac-Z production. If this indeed occurs, cells expressing an H-2K^{k} binding peptide will also be activated by an anti-H-2K^{d} mAb, and vice-versa. As shown in FIG. 3, this is really the case. This finding implies to an elevated pool of membranal β₂m, which can become available by lateral diffusion for binding to their cognate MHC class I alleles following dissociation of their original peptide.

### Example 2

### Construction and Expression of dcβ₂m Molecules Harboring Antigenic Peptides of the B16 Mouse Melanoma Model

The APCs for the animal studies are based on the commonly used RMA and RMA-S H-2^{b} cell lines. In the animal experiments, focus is on a mouse melanoma expressing a natural K^{b}-restricted, TAA-derived peptide, and, as a control for peptide specificity, a derivative of the same mouse melanoma is employed presenting another, highly immunogenic K^{b}-restricted peptide, following DNA transfection.

B16 is a spontaneous murine (m) melanoma originating in C57BL/6 mice. B16-F10.9 is a high metastatic line of B16, which shows a low cell surface expression of H-2K^{b}, and K1 is a low metastatic B16 variant, expressing high level of H-2K^{b} following DNA transfection (Porgador et al., 1989). TRP-2 was recently identified as a tumor rejection antigen for the B16 melanoma (Bloom et al., 1997). TRP-2₁₈₁₋₁₈₈, (VYDFFVWL—the peptide of SEQ ID NO: 43, in which the residue S at the amino terminal is absent) is a K^{b}-restricted peptide from TRP-2, and is a major peptide epitope in the induction of tumor-reactive CTLs, which mediate tumor rejection. MO5 is a chicken ovalbumin-transfected variant of the B16 melanoma. It presents the peptide OVA₂₅₇₋₂₆₄ (SIINFEKL—SEQ ID NO: 48), possessing H-2K^{b} anchor residues F at position 5 and L at position 8) in the context of K^{b}.

For further studies, including the B16 model, we replaced both the peptide bridge and the transmembrane and cytoplasmic domains of membranal β₂m with those of the H-2K^{b} molecule. A new XhoI/NotI fragment (see FIG. 1), encoding this polypeptide stretch, was produced, bearing the DNA sequence of SEQ ID NO: 49:

gag ccc tcg agc tcc act gtc tcc aac atg gcg acc gtt gct gtt ctg gtt gtc ctt gga gct gca ata gtc act gga gct gtg gtg gct ttt gtg atg aag atg aga agg aga aac aca ggt gga aaa gga ggg gac tat gct ctg gct cea ggc tcc cag acc tct gat ctg tct ctc cca gat tgt aaa gtg atg gtt cat gac cct cat tct cta gcg tga.

From the 11^{th} codon (gcg) till the end this sequence encompasses the intact transmembrane and cytoplasmic portion of H-2K^{b} (positions 658-852 in GenBank accession J00400). The bridge is LRWEPSSSTVSNM (SEQ ID NO: 50), a fusion between the connecting peptide of HLA-A2 (at the carboxyl terminal) and H2-K^{b}. It is encoded by the sequence ctg aga tgg gag ccC TCG AGc tcc act gtc tcc aac atg, (SEQ ID NO: 51) with an XhoI site incorporated into the sequence.

The sequence of the sense primer comprises 2b protection and an XhoI site followed by the 3′ part of the H-2K^{b} connecting peptide”

(SEQ ID NO: 52)

5′ CCC TCG AGC TCC ACT GTC TCC AAC ATG GCG 3′

The sequence of the reverse primer comprises 3b protection, a NotI site and it corresponds to GenBank accession J00400 positions 858-875:

(SEQ ID NO: 53)

5′ CGC GCGG CCGC AAG TCC ACT CCA GGC AGC 3′

The fragment was produced by RT-PCR performed on mRNA prepared from RMA (H-₂^{b}) cells.

The sequences encoding both Trp-2₁₈₁₋₁₈₈ and OVA₂₅₇₋₂₆₄ were cloned as XbaI/BamHI fragments (see FIG. 1) with synthetic oligonucleotides, which were used for PCR amplification of the gene segments encoding mβ₂m leader peptide.

The sequence of the sense primer is:

(SEQ ID NO: 54)

5′ GCG TCT AGA GCT TCA GTC GTC AGC ATG GCT CGC 3′

It comprises 3b protection, an XbaI site and positions 38-61 in GenBank accession X01838, composed of 15 b 5′ non-translated region of mβ₂m leader and the first 3 leader codons, including the ATG.

The sense sequence of the reverse primer for TRP-2₁₁₈₋₁₈₈ is:

(SEQ ID NO: 55)

5′ CTG ACC GGC TTG TAT GCT GTG TAT GAC TTT TTT GTG
TGG CTC GGA GGT GGC GGA TCC GCG 3′

It corresponds to the last 6 codons of the mβ₂m leader, the 8 codons for TRP-2₁₈₁₋₁₈₈ (GBA X66349 945-968), the first 5 codons of the linker peptide and 3b protection.

The final (reverse complementary sequence) is:

(SEQ ID NO: 56)

5′ CGC GGA TCC GCC ACC TCC GAG CCA CAC AAA AAA GTC
ATA CAC AGC ATA CAA GCC GGT CAG 3′

The sense sequence of the reverse primer for OVA₂₅₇₋₂₆₄ is:

(SEQ ID NO: 57)

5′ CTG ACC GGC TTG TAT GCT AGT ATA ATC AAC TTT GAA
AAA CTG GGA GGT GGC GGA TCC GCG 3′

It corresponds to the last 6 codons of the mβ₂m leader, the 8 codons for the OVA₂₅₇₋₂₆₄ (GenBank accession J00895, positions 7870-7893), the first 5 codons of the linker peptide and 3b protection.

The final (reverse complementary) sequence is:

(SEQ ID NO: 58)

5′ CGC GGA TCC GCC ACC TCC CAG TTT TTC AAA GTT GAT
TAT ACT AGC ATA CAA GCC GGT CAG 3′

As a BamHI/XhoI fragment encoding the carboxyl terminal of the linker peptide, the full mature hβ₂m and the amino terminal of the bridge we used the same fragment described in WO 01/91698. We created a similar fragment encoding mβ₂m, with the sequence of SEQ ID NO: 59:

gga tcc gga ggt ggt tct ggt gga ggt tcg atc cag aaa acc cct caa att caa gta tac tca cgc cac cca ccg gag aat ggg aag ccg aac ata ctg aac tgc tac gta aca cag ttc cac ccg cct cac att gaa atc caa atg ctg aag aac ggg aaa aaa att cct aaa gta gag atg tca gat atg tcc ttc agc aag gac tgg tct ttc tat atc ctg gct cac act gaa ttc acc ccc act gag act gat aca tac gcc tgc aga gtt aag cat gac agt atg gcc gag ccc aag acc gtc tac tgg gat cga gac atg ctg aga tgg gag ccc tcg agc

From the 11^{th} codon (atc) till the 8^{th} codon before the end (atg) it encompasses positions 113-409 in GenBank accession X01838.

The sequence of the sense primer is:

(SEQ ID NO: 60)

5′ GCG GGA TCC GGA GGT GGT TCT GGT GGA GGT TCG ATC
CAG AAA ACC CCT CAA ATT C 3′

It comprises 3b protection, a BamHI site, a segment encoding the carboxyl terminal of the linker peptide and the first 7 codons of the mature mβ₂m.

The sequence of the reverse primer is:

(SEQ ID NO:61)

5′ GCG GCT CGA GGG CTC CCA TCT CAG CAT GTC TCG ATC
CCA GTA GAC 3′

It comprises 4b protection, an XhoI site and it corresponds the last 7 codons of mβ₂m and to the amino terminal part of the bridge.

RT-PCR for amplification of mβ₂m sequences was performed on mRNA prepared from MD45 cells. Following verification of DNA sequences, each of the two XbaI/BamHI fragments was cloned into either pCI-Neo or pBJ1-Neo expression vectors, together with the BamHI/XhoI and the XhoI/NotI fragments described herein.

Stable transfectants with the resulting plasmids were generated and are listed in Table 1.

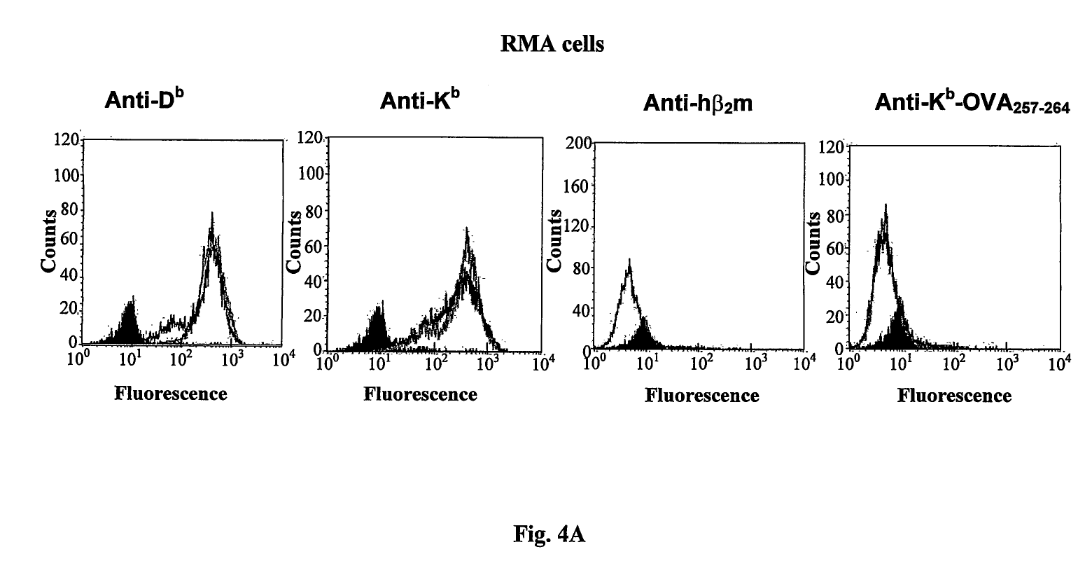

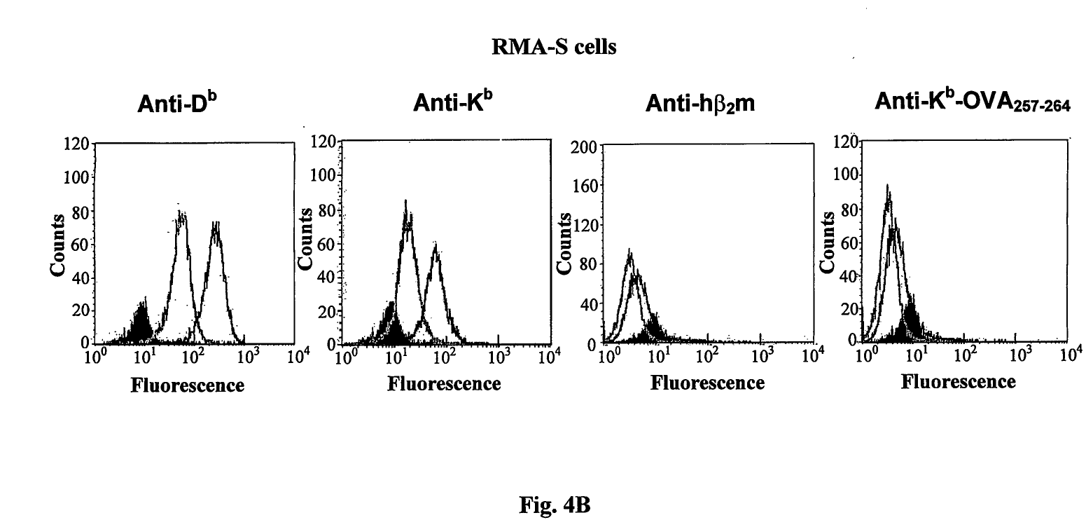

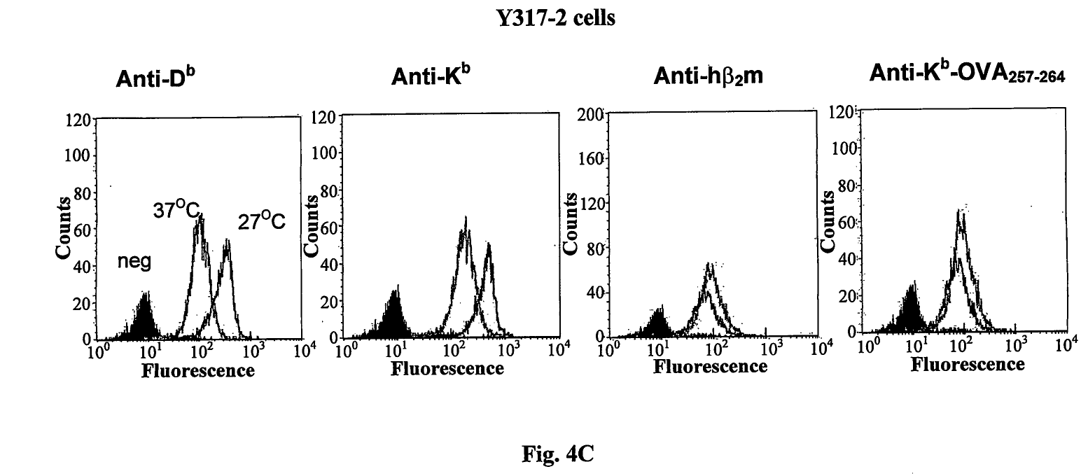

In order to evaluate expression of the new dcβ₂m constructs, we performed the FACS analysis shown in FIG. 4. In this experiment, we compared expression of different MHC class I components on RMA, RMA-S and Y317-2 cells (transfected with OVA₂₅₇₋₂₆₄ fused to membranal hβ₂m), both at 37° C. and 27° C. At this lower temperature MHC class I molecules on the TAP-deficient RMA-S cells are stabilized and their cell surface level increases. It is evident from this analysis that level of both H-2K^{b} and H-2D^{b} is considerably higher in Y317-2 cells than in their parental RMA-S cells. These results support our previous ones (FIG. 3) in showing that the chimeric polypeptide can associate on the cell surface with allelic products (in this case H-2D^{b}) other than the one binding the encoded antigenic peptide (K^{b}) Surface expression of the antigenic K^{b}/OVA₂₅₇₋₂₆₄ complex (as judged by staining with the 25D-1.16 mAb) conclusively indicates that presentation is TAP-independent. Comparison of mean fluorescence intensity (MFI) of expression at 37° C. is presented in Table 3 and reveals 57% H-2K^{b} occupancy in the transfectant.

TABLE 3
Mean fluorescence intensities of clone Y317-2 stained with an
allele-specific and complex-specific mAbs.

Antibody

| Cell | Anti-H-2K^{b} | 25D-1.16 |
|---|---|---|
| Y317-2 | 121.7 | 69.7 |

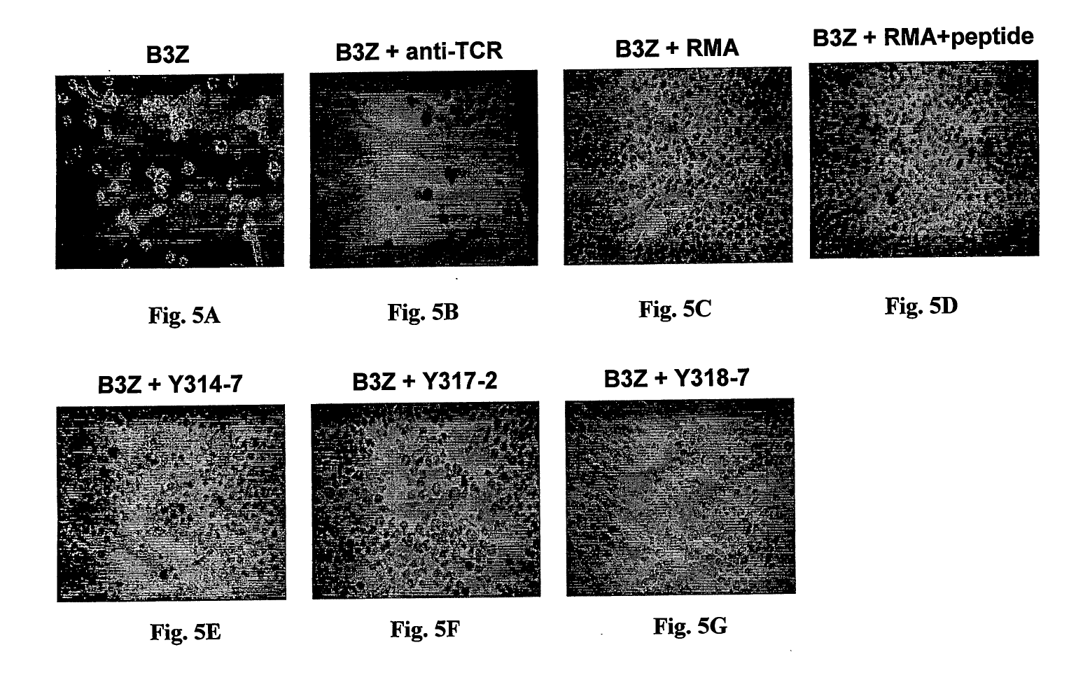

We then went on to confirm that the linker peptide, which joins the carboxyl terminal of the antigenic peptide to the amino terminal of β₂m, does not interfere with T cell recognition. To this end we examined specific activation of B3Z, an H-2K^{b}-restricted, OVA₂₅₇₋₂₆₄-specific CTL hybridoma, by RMA-S clones expressing dcβ₂m with OVA₂₅₇₋₂₆₄. The results, presented in FIG. 5, show that the level of activation is indistinguishable from that achieved following incubation of parental RMA-S cells with synthetic OVA₂₅₇₋₂₆₄ and rule out major disruption of TCR-ligand interaction in this case.

### Example 3

### Evaluating Contribution of Membrane Anchorage of β₂m to MHC Class I Stability

In the experimental system described in WO 01/91698, a plasmid was assembled, designated 21-2, which encodes a membranal hβ₂m, linked to the transmembrane and cytoplasmic region of mouse CD3 ζ chain. Another plasmid, 323-3 was assembled, in which the CD3 ζ portion was replaced with those of H-2K^{b}. This was done as follows:

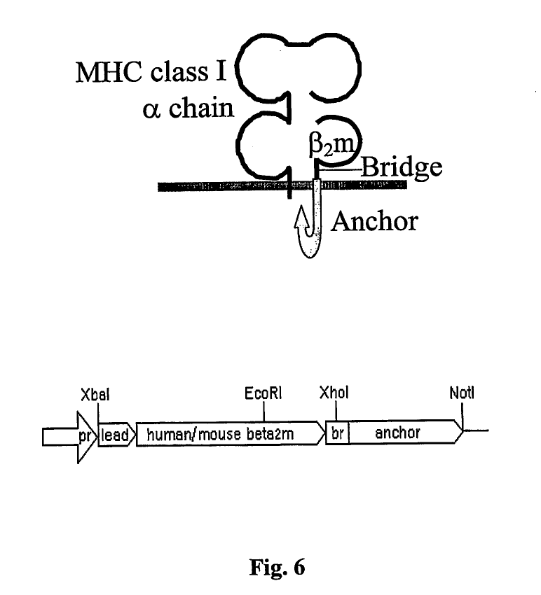

Scheme of genetic constructs encoding these single chimeric β₂m (scβ₂m) derivatives and of their expected polypeptide products associated with an MHC class I heavy chain are illustrated in FIG. 6.

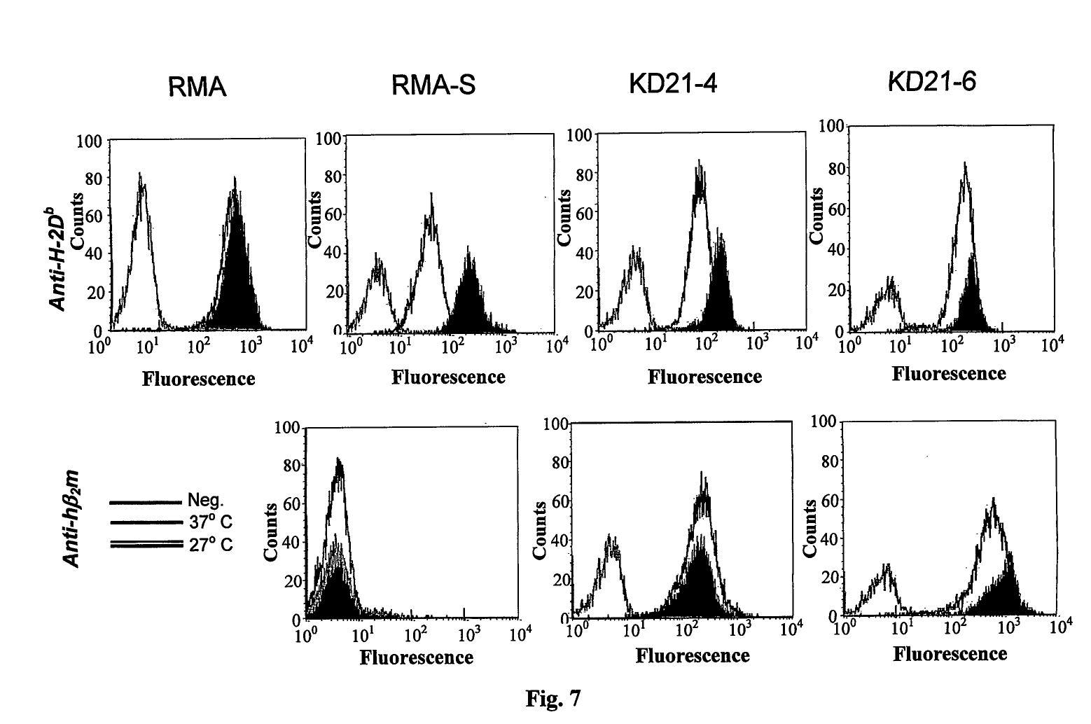

Plasmid 21-2 was introduced into RMA-S cells. Following FACS analysis of G418-resistant transfectants with the anti-hβ₂m antibody, two clones, designated KD21-4 and KD21-6, were chosen, the latter expressing higher level of membranal β₂m. These two clones were analyzed for the ability of the scβ₂m product to stabilize the MHC class I molecule H-2D^{b} at 37° C. Results of a typical experiment are presented in FIG. 7. It is clear from these results that H-2D^{b} level is elevated at 37° C. compared with the parental RMA-S cells, and that this elevation correlates with expression level of hβ₂m. In fact, for KD21-6, the level of surface H-2D^{b} is comparable to that of the wild-type RMA cells.

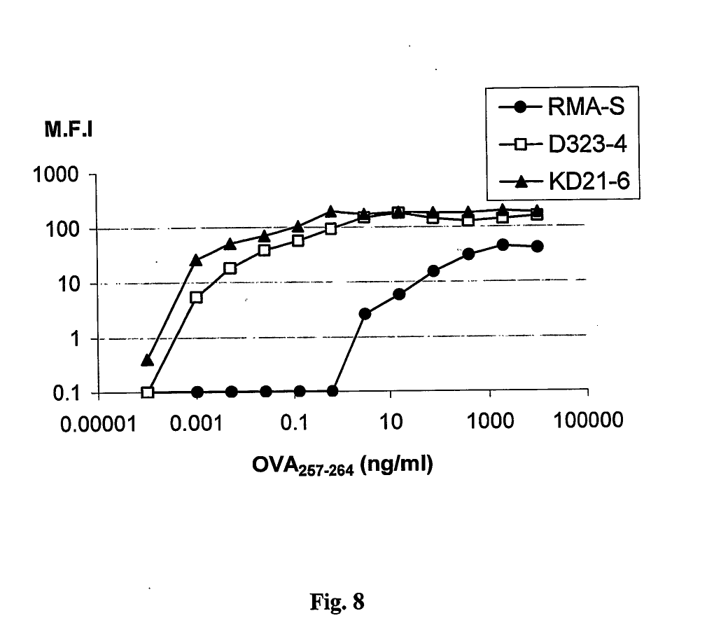

Plasmid 323-3 was similarly introduced to RMA-S cells and a stable transfectant, designated D323-4, which expresses high level of hβ₂m, was selected. In the next experiment we evaluated the ability of both KD21-6 and D323-4 transfectants to bind exogenously added synthetic OVA₂₅₇₋₂₆₄ peptide through H-2K^{b}, in comparison with parental RMA-S cells, exploiting the complex-specific 25D-1.16 mAb. This experiment was repeated 6 times, producing essentially identical results. Results of one of these experiments are shown in FIG. 8. They demonstrate approximately 3 logs enhancement of the ability to bind exogenous peptide, while maximal level of binding increases only 3-4-fold compared with RMA-S cells. These findings imply that expression of the scβ₂m products results in a vast enhancement in the functional affinity of the antigenic peptide to the MHC class I molecule. It should be noted that the nature of the β₂m anchor (CD3 ζ in KD21-6 or H-2K^{b} in D323-4) has little influence on the magnitude of this striking phenomenon.

### Example 4

### In-vivo Assessment of dcβ₂m-Based APCs

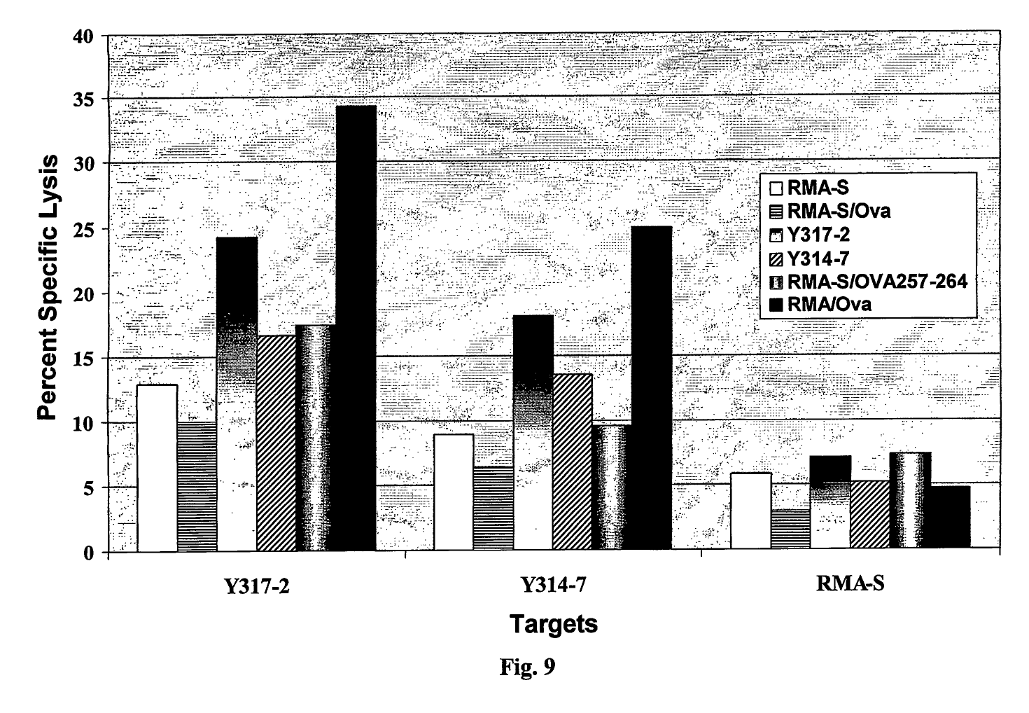

For in vivo evaluation of the capacity of dcβ₂m-based APCs to induce a specific CTL response, the RMA-S transfectants Y317-2 and Y314-7, expressing OVA₂₅₇₋₂₆₄ linked to hβ₂m or mβ₂m, respectively, were compared with cells exogenously loaded by peptides. In a preliminary experiment, C57BL/6 (B6) mice were immunized with the indicated cells. CTLs prepared from immunized mice were used in a cell cytotoxicity assay, in which transfectants were evaluated as target cells at various effector/target ratios. Results are depicted in FIG. 9 and indicate that both Y317-2 and Y314-7 cells can serve as immunogens and as target cells for CTLs. These finding reinforce our previous conclusion that dcβ₂m is an efficient vehicle for presentation of pre-selected antigenic peptides and that the linker peptide does not interfere with T cell recognition.

### Example 5

### Assembly and Preliminary Evaluation of β₂m Fused to CD40 Transmembrane and Cytoplasmic Region

DC licensing requires engagement of the CD40L on the CD4 T cell with CD40 on the DC and is a mandatory step in the elicitation of many CTL responses. We reasoned that supplementing β₂m with the intracellular portion of CD40 might trigger CD40 signaling upon encounter of DCs expressing these new dcβ₂m constructs with specific CTLs, circumventing CD4 T cell help. In other words, the CD40 signaling moiety can serve as an adjuvant in membranal β₂m-based vaccines. To test this idea we assembled a new scβ₂m expression plasmid (encoding hβ₂m, according to the general scheme illustrated in FIG. 6), in which the encoded anchor comprises CD40 transmembrane and cytoplasmic portion. This was done as follows:

The bridge is LRWEPSSSTVSNM (SEQ ID NO:50), a fusion between the connecting peptide of H-K^{b} with that of HLA-A2, as in Example 2. The gene segment encoding mouse CD40 transmembrane and cytoplasmic region encompasses positions 588-878 in GenBank accession M83312 and its DNA sequence (SEQ ID NO: 62) is:

gcc ctg ctg gtc att cct gtc gtg atg ggc atc ctc atc acc att ttc ggg gtg ttt ctc tat atc aaa aag gtg gtc aag aaa cca aag gat aat gag atg tta ccc cct gcg gct cga cgg caa gat ccc cag gag atg gaa gat tat ccc ggt cat aac acc gct gct cca gtg cag gag aca ctg cac ggg tgt cag cct gtc aca cag gag gat ggt aaa gag agt cgc atc tca gtg cag gag cgg cag gtg aca gac agc ata gcc ttg agg ccc ctg gtc tga.

The sequence of the sense primer (SEQ ID NO: 63) is:

5′ CCC TCG AGC TCC ACT GTC TCC AAC ATG GCC CTG CTG
GTC ATT CCT G 3′.

It comprises 2b protection, an XhoI site followed by the 3′ part of the segment encoding the bridge and the first 19b encoding the CD40 portion.

The sequence of the reverse primer (SEQ ID NO: 64) is:

5′ CGC GCG GCC GCG GTC AGC AAG CAG CCA TC 3′

It corresponds to a stretch downstream the CD40 stop codon (positions 901-918 in GenBank Accession M833 12) and contains NotI and 3b protection.

Messenger RNA was prepared from the murine B cell lymphoma A20, known to express CD40, and RT-PCR was performed with the two primers. The 369 bp product was cloned into pGEMT and DNA sequence was confirmed. The XhoI-NotI fragment was excised and inserted into the expression vector pBJ1-Neo cut with XbaI and NotI, together with the XbaI-XhoI fragment from plasmid 21-2, encoding hβ₂m with its leader peptide and the amino terminal of the bridge.

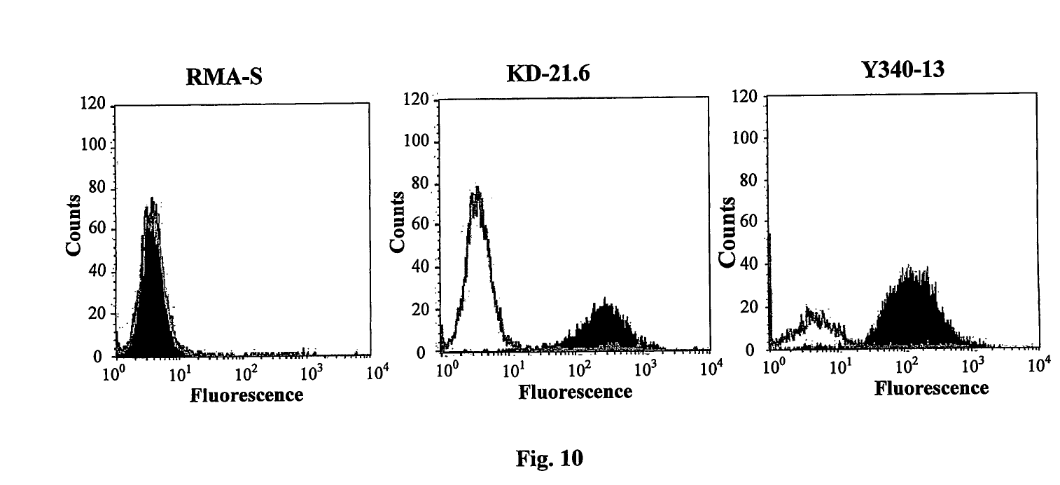

In order to assess function of the CD40 domains, we took advantage of the finding that CD40 can activate the nuclear factor of activated T cells (NFAT) (Choi et al., 1994). Plasmid DNA was introduced into B3Z cells (capable of high LacZ expression following stimulation through the NFAT-LacZ reporter gene) and resulting clones were screened for hβ₂m expression. FACS analysis, shown in FIG. 10, reveals high expression of hβ₂m in one of the transfectants (Y340-13) but none in the parental B3Z cells.

### REFERENCES

Andersen, P. S., Stryhn, A., Hansen, B. E., Fugger, L., Engberg, J., and Buus, S. 1996. A recombinant antibody with the antigen-specific, major histocompatibility complex-restricted specificity of T cells. Proc Natl Acad Sci U S A 93:1820-4.

Armstrong, T. D., Pulaski, B. A., and Ostrand_Rosenberg, S. 1998. Tumor antigen presentation: changing the rules. Cancer Immunol. Immunother. 46:70-4.

Benton, P. A. and Kennedy, R. C. 1998. DNA vaccine strategies for the treatment of cancer. Curr Top Microbiol Immunol 226:1-20.

Berger, C. L., Longley, B. J., Imaeda, S., Christensen, I., Heald, P., and Edelson, R. L. 1998. Tumor-specific peptides in cutaneous T-cell lymphoma: association with class I major histocompatibility complex and possible derivation from the clonotypic T-cell receptor. Int J Cancer 76:304-11.

Bloom, M. B., Perry-Lalley, D., Robbins, P. F., Li, Y., el-Gamil, M., Rosenberg, S. A., and Yang, J. C. 1997. Identification of tyrosinase-related protein 2 as a tumor rejection antigen for the B16 melanoma. J Exp Med 185:453-9.

Bubenik, J. 2001. Genetically engineered dendritic cell-based cancer vaccines (review). Int J Oncol. 18:475-8.

Chen, W., Rains, N., Young, D., and Stubbs, R. S. 2000. Dendritic cell-based cancer immunotherapy: potential for treatment of colorectal cancer? J Gastroenterol Hepatol 15:698-705.

Choi, M. S., Brines, R. D., Holman, M. J., and Klaus, G. G. 1994. Induction of NFAT in normal B lymphocytes by anti-immunoglobulin or CD40 ligand in conjunction with IL-4. Immunity 1: 179-87.

Cohen, I. R. and Weiner, H. L. 1988. T-cell vaccination. Immunol Today 9:332-5.

Dalgleish, A. G. 2001. Current problems in the development of specific immunotherapeutic approaches to cancer. J. Clin. Pathol. 54:675-6.

Feldman M. and Eisenbach L. 1991. MHC class I genes controlling the metastatic phenotype of tumor cells. Semin Cancer Biol. October;2(5):337-46:

Gervois, N., Guilloux, Y., Diez, E., and Jotereau, F. 1996. Suboptimal activation of melanoma infiltrating lymphocytes (TIL) due to low avidity of TCR/MHC-tumor peptide interactions. J. Exp. Med. 183:2403-7.

Gilboa, E., Nair, S. K., and Lyerly, H. K. 1998. Immunotherapy of cancer with dendritic-cell-based vaccines. Cancer Immunol, Immunother 46:82-7.

Gilboa, E. 1999. The makings of a tumor rejection antigen. Immunity 11:263-70.

Gong, J., Chen, D., Kashiwaba, M., and Kufe, D. 1997. Induction of antitumor activity by immunization with fusions of dendritic and carcinoma cells. Nature Medicine 3:558-61.

Gong, J., Nikrui, N., Chen, D., Koido, S., Wu, Z., Tanaka, Y., Cannistra, S., Avigan, D., and Kufe, D. 2000. Fusions of human ovarian carcinoma cells with autologous or allogeneic dendritic cells induce antitumor immunity. J. Immunol 165:1705-11.

Guermonprez, P., Valladeau, J., Zitvogel, L., Thery, C., and Amigorena, S. 2002. Antigen presentation and T cell stimulation by dendritic cells. Annu Rev Immunol 20:621-67.

Gurunathan, S., Klinman, D. M., and Seder, R. A. 2000. DNA vaccines: immunology, application, and optimization*. Annu Rev Immunol 18:927-74.

Hadzantonis, M. and O'Neill, H. 1999. Review: dendritic cell immunotherapy for melanoma. Cancer Biother Radiopharm. 14:11-22.

Heath, W. R. and Carbone, F. R. 2001. Cross-presentation, dendritic cells, tolerance and immunity. Annual Review of Immunology 19:47-64.

Horig, H., Young, A. C., Papadopoulos, N. J., DiLorenzo, T. P., and Nathenson, S. G. 1999. Binding of longer peptides to the H-2Kb heterodimer is restricted to peptides extended at their C terminus: refinement of the inherent MHC class I peptide binding criteria. J Immunol 163:4434-41.

Jager, E., Jager, D., and Knuth, A. 2002. Clinical cancer vaccine trials. Curr Opin Immunol 14:178-82.

Janeway, C. A. and Bottomly, K. 1994. Signals and signs for lymphocyte responses. Cell 76:275-85.

Kaufmann, Y., Berke, G., and Eshhar, Z. 1981. Cytotoxic T lymphocyte hybridomas that mediate specific tumor-cell lysis in vitro. Proc Natl Acad Sci U S A 78:2502-6.

Kumar, V., Tabibiazar, R., Geysen, H. M., and Sercarz, E. 1995. Immunodominant framework region 3 peptide from TCR V beta 8.2 chain controls murine experimental autoimmune encephalomyelitis. J Immunol 154:1941-50.

Kumar, V. and Sercarz, E. 2001. An integrative model of regulation centered on recognition of TCR peptide/MHC complexes. Immunol Rev 182:113-21.

Levitsky, V., Zhang, Q. J., Levitskaya, J., and Masucci, M. G. 1996. The life span of major histocompatibility complex-peptide complexes influences the efficiency of presentation and immunogenicity of two class I-restricted cytotoxic T lymphocyte epitopes in the Epstein-Barr virus nuclear antigen 4. Jf Exp Med 183:915-26.

Lone, Y. C., Motta, I., Mottez, E., Guilloux, Y., Lim, A., Demay, F., Levraud, J. P., Kourilsky, P., and Abastado, J. P. 1998. In vitro induction of specific cytotoxic T lymphocytes using recombinant single-chain MHC class I/peptide complexes. J Immunother 21:283-94.

Minev, B. R., Chavez, F. L., and Mitchell, M. S. 1999. Cancer vaccines: novel approaches and new promise. Pharmacol and Therapeut 81:121-39.

Moingeon, P.2001. Cancer vaccines. Vaccine 19:1305-26.

Mottez, E., Langlade_Demoyen, P., Gournier, H., Martinon, F., Maryanski, J., Kourilsky, P., and Abastado, J. P. 1995. Cells expressing a major histocompatibility complex class I molecule with a single covalently bound peptide are highly immunogenic. J Exp Med 181:493-502.

Nouri-Shirazi, M., Banchereau, J., Fay, J., and Palucka, K. 2000. Dendritic cell based tumor vaccines. Immunol Letters 74:5-10.

Offner, H., Jacobs, R., Bebo, B. F., Jr., and Vandenbark, A. A. 1999. Treatments targeting the T cell receptor (TCR): effects of TCR peptide-specific T cells on activation, migration, and encephalitogenicity of myelin basic protein-specific T cells. Springer Semin Immunopathol 21:77-90.

Ojcius, D. M., Langlade-Demoyen, P., Gachelin, G., and Kourilsky, P. 1994. Role for MIC class I molecules in selecting and protecting high affinity peptides in the presence of proteases. J Immunol 152:2798-810.

Porgador, A., Snyder, D., and Gilboa, E. 1996. Induction of antitumor immunity using bone marrow-generated dendritic cells. J Immunol 156:2918-26.

Porgador, A., Feldman, M., and Eisenbach, L. 1989. H-2Kb transfection of B16 melanoma cells results in reduced tumourigenicity and metastatic competence. J Immunogen 16:291-303.

Porgador, A., Yewdell, J. W., Deng, Y., Bennink, J. R., and Germain, R. N. 1997. Localization, quantitation, and in situ detection of specific peptide-MHC class I complexes using a monoclonal antibody. Immunity 6:715-26.

Porgador, A., Irvine, K. R., Iwasaki, A., Barber, B. H., Restifo, N. P., and Germain, R. N. 1998. Predominant role for directly transfected dendritic cells in antigen presentation to CD8+T cells after gene gun immunization. J Exp Med 188:1075-82.

Reis e Sousa, C. 2001. Dendritic cells as sensors of infection. Immunity 14:495-8.

Rosenberg, S. A. 1999. A new era of cancer immunotherapy: converting theory to performance. CA Cancer J Clin. 49:70-3, 65.

Sanderson, S. and Shastri, N. 1994. LacZ inducible, antigen/MHC-specific T cell hybrids. Int Immunol 6:369-76.

Saric, T., Chang, S. C., Hattori, A., York, I. A., Markant, S., Rock, K. L., Tsujimoto, M., and Goldberg, A. L. 2002. An IFN-gamma-induced aminopeptidase in the ER, ERAP1, trims precursors to MHC class I-presented peptides. Nat Immunol 3:1169-76.

Serwold, T., Gonzalez, F., Kim, J., Jacob, R., and Shastri, N. 2002. ERAAP customizes peptides for MHC class I molecules in the endoplasmic reticulum. Nature 419:480-3.

Sherritt, M., Cooper, L., Moss, D. J., Kienzle, N., Altman, J., and Khanna, R. 2001. Immunization with tumor-associated epitopes fused to an endoplasmic reticulum translocation signal sequence affords protection against tumors with down-regulated expression of MHC and peptide transporters. Int Immunol 13:265-71.

Snyder, H. L., Yewdell, J. W., and Bennink, J. R. 1994. Trimming of antigenic peptides in an early secretory compartment. J Exp Med 180:2389-94.

Snyder, H. L., Bacik, I., Yewdell, J. W., Behrens, T. W., and Bennink, J. R. 1998. Promiscuous liberation of MHC-class I-binding peptides from the C termini of membrane and soluble proteins in the secretory pathway. Eur J Immunol 28:1339-46.

Sogn, J. A. 1998. Tumor immunology: the glass is half full. Immunity 9:757-63.

Sogn, J. A. 2000. The status of tumor immunology and cancer immunotherapy. Introduction. Immunol Investig 29:81-4.

Steinman, R. M. 1989. Dendritic cells: clinical aspects. Res Immunol 140:911-8; discussion 918-26.

Stryhn, A., Pedersen, L. O., Hohm, A., and Buus, S. 2000. Longer peptide can be accommodated in the MHC class I binding site by a protrusion mechanism. Eur J Immunol 30:3089-99.

Tafuro, S., Meier, U. C., Dunbar, P. R., Jones, E. Y., Layton, G. T., Hunter, M. G., Bell, J. I., and McMichael, A. J. 2001. Reconstitution of antigen presentation in HLA class I-negative cancer cells with peptide-beta2m fusion molecules. Eur J Immunol 31:440-9.

Takahashi, H., Nakagawa, Y., Yokomuro, K., and Berzofsky, J. A. 1993. Induction of CD8+ cytotoxic T lymphocytes by immunization with syngeneic irradiated HIV-1 envelope derived peptide-pulsed dendritic cells. Int Immunol 5:849-57.

Tirosh, B., el_Shami, K., Vaisman, N., Carmon, L., Bar_Haim, E., Vadai, E., Feldman, M., Fridkin, M., and Eisenbach, L. 1999. Immunogenicity of H-2Kb-low affinity, high affinity, and covalently-bound peptides in anti-tumor vaccination. Immunol Letters 70:21-8.

Tsomides, T. J., Aldovini, A., Johnson, R. P., Walker, B. D., Young, R. A., and Eisen, H. N. 1994. Naturally processed viral peptides recognized by cytotoxic T lymphocytes on cells chronically infected by human immunodeficiency virus type 1. J Exp Med 180:1283-93.

Uger, R. A. and Barber, B. H. 1998. Creating CTL targets with epitope-linked beta 2-microglobulin constructs. J Immunol 160:1598-605.

Uger, R. A., Chan, S. M., and Barber, B. H. 1999. Covalent linkage to beta2-microglobulin enhances the MHC stability and antigenicity of suboptimal CTL epitopes. J Immunol 162:6024-8.

Vora, A. R., Rodgers, S., Parker, A. J., Start, R., Rees, R. C., and Murray, A. K. 1997. An immunohistochemical study of altered immunomodulatory molecule expression in head and neck squamous cell carcinoma. Brit J Cancer 76:836-44.

Watson, G. A. and Lopez, D. M. 1995. Aberrant antigen presentation by macrophages from tumor-bearing mice is involved in the down-regulation of their T cell responses. J Immunol 155:3124-34.

Wen, Y. J. and Lim, S. H. 1997. T cells recognize the VH complementarity-determining region 3 of the idiotypic protein of B cell non-Hodgkin's lymphoma. Eur J Immunol 27:1043-7.

White, J., Crawford, F., Fremont, D., Marrack, P., and Kappler, J. 1999. Soluble class I MHC with beta2-microglobulin covalently linked peptides: specific binding to a T cell hybridoma. J Immunol 162:2671-6.

York, I. A., Chang, S. C., Saric, T., Keys, J. A., Favreau, J. M., Goldberg, A. L., and Rock, K. L. 2002. The ER aminopeptidase ERAP1 enhances or limits antigen presentation by trimming epitopes to 8-9 residues. Nat Immunol 3:1177-84.

Zhang, W., Cao, X., and Huang, X. 1997. [In vivo induction of antitumor immune response by tumor cells fused with GM-CSF gene-modified dendritic cells]. Zhonghua Yi Xue Za Zhi. (Chinese). 77:39-42.

1. A polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polypeptide stretch that allows the anchorage of the β2-microglobulin molecule to the cell membrane, and through its amino terminal to at least one antigenic peptide comprising a MHC class I epitope, wherein said antigenic peptide is not related to an autoimmune disease.

2. The polynucleotide of claim 1, wherein said polypeptide stretch at the β2-microglobulin carboxyl terminal consists of a bridge peptide which spans the whole distance to the cell membrane, said bridge peptide being linked to a sequence which can exert the required anchoring function.

3. The polynucleotide of claim 2, wherein said bridge peptide is the peptide of SEQ ID NO: 1, of the sequence: LRWEPSSQPTIPI.

4. The polynucleotide of claim 2, wherein said bridge peptide is linked to the full or partial transmembrane and/or cytoplasmic domain of a molecule selected from the group consisting of: (i) a human MHC class I molecule selected from an HLA-A, HLA-B or HLA-C molecule; (ii) a costimulatory B7.1, B7.2 OR CD40 molecule; and (iii) a signal transduction element capable of activating T cells or antigen-presenting cells.

5. The polynucleotide of claim 4, wherein said bridge peptide is linked to the transmembrane and cytoplasmic domains from the MHC class I heavy chain HLA-A2 molecule, of the SEQ ID NO: 2, of the sequence: VGIIAGLVLFGAVITGAVVAAVMWRRKSSDRKGGSYSQAASSDSAQGSDVSLTACK V

6. The polynucleotide of claim 4 wherein said transduction element capable of activating T cells is selected from the group consisting of a component of T-cell receptor CD3, a B cell receptor polypeptide, and an Fc receptor polypeptide.

7. The polynucleotide of claim 6, wherein said component of T-cell receptor CD3 is the zeta (ζ) or eta (η) polypeptide.

8. The polynucleotide of claim 6, wherein said component of T-cell receptor CD3 comprises the transmembranal and cytoplasmic regions of the human CD3 ζ polypeptide.

9. The polynucleotide of claim 2, wherein said bridge peptide is linked through its carboxyl terminal to a GPI-anchor sequence.

10. The polynucleotide of claim 9, wherein said GPI-anchor is a peptide of SEQ ID NO: 3, of the sequence: FTLTGLLGTLVTMGLLT.

11. The polynucleotide of any one of claim 1, wherein said at least one antigenic peptide comprising a MHC class I epitope is linked to the β2-microglobulin amino terminal through a peptide linker.

12. The polynucleotide of claim 1, wherein said at least one antigenic peptide is at least one antigenic determinant of one sole antigen.

13. The polynucleotide of claim 11, wherein said at least one antigenic peptide is at least one antigenic determinant of each one of at least two different antigens.

14. The polynucleotide of claim 12, wherein said antigen is a tumor-associated antigen (TAA).

15. The polynucleotide of claim 14, wherein said TAA is selected from the group consisting of alpha-fetoprotein, BA-46/lactadherin, BAGE, BCR-ABL fusion protein, beta-catenin, CASP-8, CDK4, CEA, CRIPTO-1, elongation factor 2, ETV6-AML1 fusion protein, G250, GAGE, gp100, HER-2/neu, intestinal carboxyl esterase, KIAA0205, MAGE, MART-1/Melan-A, MUC-1, N-ras, p53, PAP, PSA, PSMA, telomerase, TRP-1/gp75, TRP-2, tyrosinase, and uroplakin Ia, Ib, II and III.

16. The polynucleotide of claim 15, wherein said antigenic peptide is selected from the group consisting of:
(i) the alpha-fetoprotein peptide GVALQTMKQ (SEQ ID NO:4);
(ii) the BAGE-1 peptide AARAVFLAL (SEQ ID NO:5);
(iii) the BCR-ABL fusion protein peptide SSKALQRPV (SEQ ID NO:6);
(iv) the beta-catenin peptide SYLDSGIHF (SEQ ID NO:7);
(v) the CDK4 peptide ACDPHSGHFV (SEQ ID NO:8);
(vi) the CEA peptide YLSGANLNL (SEQ ID NO:9);
(vii) the elongation factor 2 peptide ETVSEQSNV (SEQ ID NO:10);
(viii) the ETV6-AML1 fusion protein peptide RIAECILGM (SEQ ID NO:11);
(ix) the G250 peptide HLSTAFARV (SEQ ID NO:12);
(x) the GAGE-1,2,8 peptide YRPRPRRY (SEQ ID NO:13);
(xi) the gp100 peptides KTWGQYWQV (SEQ ID NO:14), (A)MLGTHTMEV (SEQ ID NO:15), ITDQVPFSV (SEQ ID NO:16), YLEPGPVTA (SEQ ID NO:17), LLDGTATLRL (SEQ ID NO:18), VLYRYGSFSV (SEQ ID NO:19), SLADTNSLAV (SEQ ID NO:20), RLMKQDFSV (SEQ ID NO:21), RLPRIFCSC (SEQ ID NO:22), LIRRRLMK (SEQ ID NO:23), ALLAVGATK (SEQ ID NO:24), IALNFPGSQK (SEQ ID NO:25) and ALNFPGSQK (SEQ ID NO:26);
(xii) the HER-2/neu peptide KIFGSLAFL (SEQ ID NO:27);
(xiii) the intestinal carboxyl esterase peptide SPRWWPTCL (SEQ ID NO:28);
(xiv) the KIAA0205 peptide AEPINIQTW (SEQ ID NO:29);
(xv) the MAGE-1 peptides EADPTGHSY (SEQ ID NO:30) and SLFRAVITK (SEQ ID NO:31);
(xvi) the MAGE-3 peptides EVDPIGHLY (SEQ ID NO:32) and FLWGPRALV (SEQ ID NO:33);
(xvii) the MART-1/Melan-A peptide (E)AAGIGILTV (SEQ ID NO:34);
(xviii) the MUC-1 peptide STAPPVHNV (SEQ ID NO:35);
(xix) the N-ras peptide ILDTAGREEY (SEQ ID NO:36);
(xx) the p53 peptide LLGRNSFEV (SEQ ID NO:37);
(xxi) the PSA peptides FLTPKKLQCV (SEQ ID NO:38) and VISNDVCAQV (SEQ ID NO:39);
(xxii) the telomerase peptide ILAKFLHWL (SEQ ID NO:40);
(xxiii) the TRP-1 peptide MSLQRQFLR (SEQ ID NO:41);
(xxiv) the TRP-2 peptides LLGPGRPYR (SEQ ID NO:42), SVYDFFVWL (SEQ ID NO:43), and TLDSQVMSL (SEQ ID NO:44);
(xxv) the TRP2-INT2 peptide EVISCKLIKR (SEQ ID NO:45); and
(xxvi) the tyrosinase peptide KCDICTDEY (SEQ ID NO:46).

17. The polynucleotide of any one of claim 14, wherein said at least one antigenic peptide is at least one antigenic determinant of one sole tumor-associated antigen.

18. The polynucleotide of claim 17, wherein said at least one antigenic peptide is at least one HLA-A2 binding peptide and at least one HLA-A3 binding peptide derived from the melanoma-associated antigen gp100.

19. The polynucleotide of claim 18, wherein said at least one HLA-A2 binding peptide derived from gp100 is selected from the group consisting of SEQ ID NO: 14, 15, 16, 17, 18, 19, 20, 21 and 22, and said at least one gp100 HLA-A3 binding peptide is selected from the group consisting of SEQ ID NO: 23, 24, 25 and 26.

20. The polynucleotide of claim 14, wherein said at least one antigenic peptide is at least one antigenic determinant of each one of at least two different tumor-associated antigens.

21. The polynucleotide of claim 20, wherein said at least one antigenic peptide is at least one HLA-A2 binding peptide derived from each one of the melanoma associated antigens gp100 and Melan-A/MART-1.

22. The polynucleotide of claim 21, wherein said at least one antigenic peptide is at least one HLA-A3-restricted gp100 and at least one HLA-A2-restricted Melan-AIMART-1 peptide.

23. The polynucleotide of claim 12, wherein said antigen is an antigen from a pathogen selected from the group consisting of a bacterial, viral, fungal and parasite antigen.

24. The polynucleotide of claim 23 wherein the antigen is a viral antigen.

25. The polynucleotide of claim 24 wherein the viral antigen is an HIV protein selected from the group consisting of the HIV-1 regulatory proteins Tat and Rev and the HIV envelope protein, in which case the antigenic peptide derived therefrom has the sequence RGPGRAFVTI (SEQ ID NO:47).

26. The polynucleotide of claim 11, wherein said at least one antigenic peptide is at least one idiotypic peptide expressed by autoreactive T lymphocytes.

27. The polynucleotide of claim 26, wherein said at least one idiotypic peptide is derived from a CDR (complementarity-determining region) sequence of an immunoglobulin or of a TCR chain, optionally containing said CDR flanking segments.

28. The polynucleotide of claim 27, wherein said CDR is the CDR3 of an immunoglobulin or of a TCR chain.

29. The polynucleotide of claim 1 that is an expression vector.

30. An expression vector comprising a polynucleotide according to claims 1.

31. A recombinant viral vector of claim 30.

32. An antigen-presenting cell transfected with a polynucleotide comprising a sequence encoding a polypeptide comprising a β2-microglobulin molecule that is linked through its carboxyl terminal to a polypeptide stretch that allows the anchorage of the β2-microglobulin molecule to the cell membrane, and through its amino terminal to at least one antigenic peptide comprising a MHC class I epitope.

33. The antigen-presenting cell of claim 32 selected from the group consisting of a dendritic cell, a macrophage, a B cell and a fibroblast.

34. The antigen-presenting cell of claim 32 wherein said antigenic peptide is a peptide not related to an autoimmune disease.

35. The antigen-presenting cell of claim 34, wherein said antigenic peptide is at least one peptide derived from at least one TAA.

36. The antigen-presenting cell of claim 34, wherein said antigenic peptide is at least one peptide derived from an antigen from a pathogen selected from the group consisting of a bacterial, a viral, a fungal and a parasite antigen.

37. A DNA vaccine comprising a polynucleotide of claims 1 or an expression vector of claim 30.

38. The DNA vaccine of claim 37 for prevention or treatment of cancer wherein said polynucleotide is a polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polypeptide stretch that allows the anchorage of the β2-microglobulin molecule to the cell membrane, and through its amino terminal to at least one antigenic peptide comprising a MHC class I epitope, and said at least one antigenic peptide is at least one antigenic determinant of one sole tumor-associated antigen (TAA).

39. The DNA vaccine of claim 37 for prevention or treatment of a disease caused by a pathogenic organism wherein said polynucleotide is a polynucleotide comprising a sequence encoding a polypeptide that is capable of high level presentation of antigenic peptides on antigen-presenting cells, wherein the polypeptide comprises a β2-microglobulin molecule that is linked through its carboxyl terminal to a polyegptide stretch that allows the anchorage of the β2-microglobulin molecule to the cell membrane, and through its amino terminal to at least one antigenic peptide comprising a MHC class I epitope, and said at least one antigenic peptide is at least one antigenic determinant of one sole antigen from a pathogen selected from the group consisting of a bacterial, viral, fungal and parasite antigen.

40. A cellular vaccine, which comprises an antigen presenting cell of claim 32.

41. The cellular vaccine of claim 40 wherein the antigen presenting cell is selected from the group consisting of a dendritic cell, a macrophage, a B cell and a fibroblast.

42. The cellular vaccine of claim 41, wherein the at least one antigenic peptide presented by the antigen presenting cell is a peptide not related to an autoimmune disease.

43. The cellular vaccine of claim 42 for prevention or treatment of cancer wherein the antigen presenting cell presents at least one peptide derived from at least one tumor associated antigen.

44. The cellular vaccine of claim 42 for prevention or treatment of a disease caused by a pathogenic organism wherein the antigen presenting cell presents at least one peptide derived from a pathogenic antigen.

45. A cellular vaccine for the prevention or treatment of cancer comprising antigen presenting cells which express a polypeptide consisting of β2-microglobulin linked through its carboxyl terminal to a polypeptide stretch that allows the anchorage of the β2-microglobulin molecule to a cell membrane, wherein said cells have been pulsed with at least one antigenic peptide derived from at least one tumor associated antigen.

46. A cellular vaccine for treatment of cancer comprising tumor cells transfected with a polynucleotide comprising a sequence encoding a polypeptide comprising a β2-microglobulin molecule that is linked through its carboxyl terminal to a polypeptide stretch that allows the anchorage of the β2-microglobulin molecule to the cell membrane.

47. A method of immunizing a mammal against a tumor-associated antigen comprising the step of immunizing the mammal with a cellular vaccine, which comprises an antigen presenting cell transfected with a polynucleotide comprising a sequence encoding a polypeptide comprising a β2-microglobulin molecule that is linked through its carboxyl terminal to a polypeptide stretch that allows the anchorage of the β2-microglobulin molecule to the cell membrane, and through its amino terminal to at least one antigenic peptide comprising a MHC class I epitope, wherein said antigen presenting cell is selected from the group consisting of a dendritic cell, a macrophage, a B cell or a fibroblast, and said at least one antigenic peptide is at least one peptide derived from at least one tumor-associated antigen.

48. A method of immunizing a mammal against a disease caused by a pathogenic organism comprising the step of immunizing the mammal with a cellular vaccine, which comprises an antigen presenting cell transfected with a polvnucleotide comprising a sequence encoding a polypentide comprising a β2-microglobulin molecule that is linked through its carboxyl terminal to a polypeptide stretch that allows the anchorage of the β2-microglobulin molecule to the cell membrane, and through its amino terminal to at least one antigenic peptide comprising a MHC class I epitope, wherein said antigen presenting cell is selected from the group consisting of a dendritic cell, a macrophage, a B cell or a fibroblast, and said at least one antigenic peptide is at least one antigenic peptide derived from a pathogen.

49. A pharmaceutical composition comprising as an active ingredient at least one polynucleotide of claims 1 and a pharmaceutically acceptable carrier.

50. The pharmaceutical composition of claim 49 wherein the polynucleotide comprises a sequence encoding a polypeptide comprising at least one antigenic peptide derived from at least one tumor associated antigen.

51. The pharmaceutical composition of claim 49 wherein the polynucleotide comprises a sequence encoding a polypeptide comprising at least one antigenic peptide derived from a pathogenic antigen.

52. A pharmaceutical composition comprising as an active ingredient at least one antigen presenting cell of claim 32, and a pharmaceutically acceptable carrier.

53. The polynucleotide of claim 13, wherein said antigen is a tumor-associated antigen (TAA).

54. A method of immunizing a mammal against a tumor-associated antigen comprising the step of immunizing the mammal with a cellular vaccine comprising antigen presenting cells which express a polypeptide consisting of β2-microglobulin linked through its carboxyl terminal to a polypeptide stretch that allows the anchorage of the β2-microglobulin molecule to a cell membrane, wherein said cells have been pulsed with at least one antigenic peptide derived from at least one tumor-associated antigen.

55. A method of immunizing a mammal against a tumor-associated antigen comprising the step of immunizing the mammal with a cellular vaccine comprising tumor cells transfected with a polynucleotide comprising a sequence encoding a polypeptide comprising a β2-microglobulin molecule that is linked through its carboxyl terminal to a polypeptide stretch that allows the anchorage of the β2-microglobulin molecule to the cell membrane.
